(19) United States
(12) Patent Application Publication
(10) Pub. No.: US 2003/0082754 A1
(43) Pub. Date: May 1, 2003

(54) Delta4 - desaturase genes and uses thereof

(76) Inventors: Pradip Mukerji, Gahanna, OH (US); Jennifer Thurmond, Columbus, OH (US); Yung-Sheng Huang, Upper Arlington, OH (US); Tapas Das, Worthington, OH (US)

Correspondence Address: Steven F. Weinstock, Abbott Laboratories, Department 377 / AP6D-2, 100 Abbott Park Road, Abbott Park, IL, 60064-6050, US

(21) Appl. No.: 09/849,199

(22) Filed: May 4, 2001

(51) Int. Cl. C12P 7/64; C12N 9/04; C12N 1/18; C07H 21/04; C12P 21/02; C12N 15/74

(52) U.S. Cl. 435/134; 435/69.1; 435/190; 435/320.1; 435/254.11; 536/23.2

(57) ABSTRACT

The subject invention relates to the identification of genes involved in the desaturation of polyunsaturated fatty acids at carbon 4 (i.e., “Δ4-desaturase”). In particular, Δ4-desaturase may be utilized, for example, in the conversion of adrenic acid to ω6-docosapentaenoic acid and in the conversion of ω3-docosapentaenoic acid to docosahexaenoic acid. The polyunsaturated fatty acids produced by use of the enzyme may be added to pharmaceutical compositions, nutritional compositions, animal feeds, as well as other products such as cosmetics.

### BACKGROUND OF THE INVENTION

1. Technical Field

The subject invention relates to the identification and isolation of genes that encodes an enzyme (i.e., Thraustochytrium aureum Δ4-desaturase) involved in the synthesis of polyunsaturated fatty acids and to uses thereof. In particular, Δ4-desaturase catalyzes the conversion of, for example, adrenic acid (22:4n-6) to ω6-docosapentaenoic acid (22:5n-6) and the conversion of ω3-docosapentaenoic acid (22:5n-3) to docosahexaenoic acid (22:6n-3). The converted products may then be utilized as substrates in the production of other polyunsaturated fatty acids (PUFAs). The product or other polyunsaturated fatty acids may be added to pharmaceutical compositions, nutritional composition, animal feeds as well as other products such as cosmetics.

2. Background Information

Desaturases are critical in the production of long-chain polyunsaturated fatty acids that have many important functions. For example, polyunsaturated fatty acids (PUFAs) are important components of the plasma membrane of a cell, where they are found in the form of phospholipids. They also serve as precursors to mammalian prostacyclins, eicosanoids, leukotrienes and prostaglandins.

Additionally, PUFAs are necessary for the proper development of the developing infant brain as well as for tissue formation and repair. In view of the biological significance of PUFAS, attempts are being made to produce them, as well as intermediates leading to their production, in an efficient manner.

A number of enzymes are involved in PUFA biosynthesis (see FIG. 1). For example, elongase (elo) catalyzes the conversion of γ-linolenic acid (GLA) to dihomo-γ-linolenic acid (DGLA) and of stearidonic acid (18:4n-3) to (n-3)-eicosatetraenoic acid (20:4n-3). Linoleic acid (LA, 18:2-Δ9,12 or 18:2n-6) is produced from oleic acid (18:1-Δ9) by a Δ12-desaturase. GLA (18:3-Δ6,9,12) is produced from linoleic acid by a Δ6-desaturase.

It must be noted that animals cannot desaturate beyond the Δ9 position and therefore cannot convert oleic acid into linoleic acid. Likewise, α-linolenic acid (ALA, 18:3-Δ9,12,15) cannot be synthesized by mammals. However, α-linolenic acid can be converted to stearidonic acid (STA, 18:4-Δ6,9,12,15) by a Δ6-desaturase (see PCT publication WO 96/13591 and The FASEB Journal, Abstracts, Part I, Abstract 3093, page A532 (Experimental Biology 98, San Francisco, Calif., Apr. 18-22, 1998); see also U.S. Pat. No. 5,552,306), followed by elongation to (n-3)-eicosatetraenoic acid (20:4-Δ8,11,14,17) in mammals and algae. This polyunsaturated fatty acid (i.e., 20:4-Δ8,11,14,17) can then be converted to eicosapentaenoic acid (EPA, 20:5-Δ5,8,11,14,17) by a Δ5-desaturase. EPA can then, in turn, be converted to ω3-docosapentaenoic acid (22:5n-3) by an elongase. Isolation of an enzyme or its encoding gene, responsible for conversion of ω3-docosapentaenoic acid to docosahexaenoic acid (22:6n-3) has never been reported. Two pathways for this conversion have been proposed (see FIG. 1 and Sprecher, H., Curr. Opin. Clin. Nutr. Metab. Care, Vol.2, p. 135-138, 1999). One of them involves a single enzyme, a Δ4-desaturase such as that of the present invention. In the n-6 pathway, dietary linoleic acid may be converted to adrenic acid through a series of desaturation and elongation steps in mammals (see FIG. 1). Production of ω6-docosapentaenoic acid from adrenic acid is postulated to be mediated by the Δ-desaturase discussed above.

Other eukaryotes, including fungi and plants, have enzymes which desaturate at carbon 12 (see PCT publication WO 94/11516 and U.S. Pat. No. 5,443,974) and carbon 15 (see PCT publication WO 93/11245). The major polyunsaturated fatty acids of animals therefore are either derived from diet and/or from desaturation and elongation of linoleic acid or α-linolenic acid. In view of these difficulties, it is of significant interest to isolate genes involved in PUFA synthesis from species that naturally produce these fatty acids and to express these genes in a microbial, plant, or animal system which can be altered to provide production of commercial quantities of one or more PUFAs.

In view of the above discussion, there is a definite need for the Δ4-desaturase enzyme, the respective gene encoding this enzyme, as well as recombinant methods of producing this enzyme. Additionally, a need exists for oils containing levels of PUFAs beyond those naturally present as well as those enriched in novel PUFAs. Such oils can only be made by isolation and expression of the Δ4-desaturase gene.

All U.S. patents and publications referred to herein are hereby incorporated in their entirety by reference.

### SUMMARY OF THE INVENTION

The present invention includes an isolated nucleotide sequence corresponding to or complementary to at least about 50% of the nucleotide sequence selected from the group consisting of SEQ ID NO: 14, SEQ ID NO: 15, SEQ ID NO: 16 and SEQ ID NO: 17.

The isolated nucleotide sequence may be represented by SEQ ID NO: 14, SEQ ID NO: 15, SEQ ID NO: 16 or SEQ ID NO: 17. These sequences may encode a functionally active desaturase which utilizes a monounsaturated or polyunsaturated fatty acid as a substrate. The sequences may be derived from, for example, a fungus such Thraustochytrium aureum.

The present invention also includes purified proteins (SEQ ID NO: 18, SEQ ID NO: 19, SEQ ID NO: 20 and SEQ ID NO: 21) encoded by the nucleotide sequences referred to above.

Additionally, the present invention includes a purified polypeptide which desaturates polyunsaturated fatty acids at carbon 4 and has at least about 50% amino acid similarity to the amino acid sequence of the purified proteins referred to directly above (i.e., SEQ ID NO: 18. SEQ ID NO: 19, SEQ ID NO: 20 or SEQ ID NO: 21).

Furthermore, the present invention also encompasses a method of producing a desaturase (i.e., Δ4). This method comprises the steps of: a) isolating the nucleotide sequence comprising SEQ ID NO: 14, SEQ ID NO: 15, SEQ ID NO: 16 or SEQ ID NO: 17; b) constructing a vector comprising: i) the isolated nucleotide sequence operably linked to ii) a promoter; and c) introducing the vector into a host cell under time and conditions sufficient for expression of the Δ4-desaturase. The host cell may be, for example, a eukaryotic cell or a prokaryotic cell. In particular, the prokaryotic cell may be, for example, E. coli, cyanobacteria or B. subtilis. The eukaryotic cell may be, for example, a mammalian cell, an insect cell, a plant cell or a fungal cell (e.g., a yeast cell such as Saccharomyces cerevisiae, Saccharomyces carlsbergensis, Candida spp., Lipomyces starkey, Yarrowia lipolytica, Kluyveromyces spp., Hansenula spp., Trichoderma spp. or Pichia spp.).

Additionally, the present invention also encompasses a vector comprising: a) a nucleotide sequence as represented by SEQ ID NO: 14, SEQ ID NO: 15, SEQ ID NO: 16 or SEQ ID NO: 17, operably linked to b) a promoter. The invention also includes a host cell comprising this vector. The host cell may be, for example, a eukaryotic cell or a prokaryotic cell. Suitable eukaryotic cells and prokaryotic cells are as defined above.

Moreover, the present invention also includes a plant cell, plant or plant tissue comprising the above vector, wherein expression of the nucleotide sequence of the vector results in production of a polyunsaturated fatty acid by the plant cell, plant or plant tissue. The polyunsaturated fatty acid may be, for example, selected from the group consisting of ω6-docosapentaenoic acid or docosahexaenoic acid. The invention also includes one or more plant oils or acids expressed by the above plant cell, plant or plant tissue.

Additionally, the present invention also encompasses a transgenic plant comprising the above vector, wherein expression of the nucleotide sequence of the vector results in production of a polyunsaturated fatty acid in seeds of the transgenic plant.

It should also be noted that the present invention encompasses a transgenic, non-human mammal whose genome comprises a DNA sequence encoding a Δ4-desaturase, operably linked to a promoter. The DNA sequence may be represented by SEQ ID NO: 14, SEQ ID NO: 15, SEQ ID NO: 16 or SEQ ID NO: 17. Additionally, the present invention includes a fluid (e.g., milk) produced by or tissue isolated from the transgenic, non-human mammal wherein the fluid or tissue comprises a detectable level of at least Δ4-desaturase.

Additionally, the present invention includes a method (i.e., “first” method) for producing a polyunsaturated fatty acid comprising the steps of: a) isolating the nucleotide sequence represented by SEQ ID NO: 14, SEQ ID NO: 15, SEQ ID NO: 16 or SEQ ID NO: 17; b) constructing a vector comprising the isolated nucleotide sequence; c) introducing the vector into a host cell under time and conditions sufficient for expression of Δ4-desaturase enzyme; and d) exposing the expressed Δ4-desaturase enzyme to a substrate polyunsaturated fatty acid in order to convert the substrate to a product polyunsaturated fatty acid. The substrate polyunsaturated fatty acid may be, for example, adrenic acid or ω3-docospentaenoic acid, and the product polyunsaturated fatty acid may be, for example, ω6-docosapentaenoic acid or docosahexaenoic acid, respectively. This method may further comprise the step of exposing the product polyunsaturated fatty acid to another enzyme (e.g., a Δ4-desaturase, an elongase or another desaturase) in order to convert the product polyunsaturated fatty acid to another polyunsaturated fatty acid (i.e., “second” method). In this method containing the additional step (i.e., “second” method), the product polyunsaturated fatty acid may be, for example, ω6-docosapentaenoic acid, and the “another” polyunsaturated fatty acid may be docosahexaenoic acid.

In another method, a substrate polyunsaturated fatty acid (e.g., eicosapentaenoic acid) may be exposed to an elongase or desaturase (e.g., MELO4 or other elongases or desaturases of significance in the biosynthetic pathway) in order to convert the substrate to a product polyunsaturated fatty acid (e.g., ω3-docosapentaenoic acid). The product polyunsaturated fatty acid may then be converted to a “final” product polyunsaturated fatty acid (e.g., docosahexaenoic acid) by exposure to the Δ4-desaturase of the present invention (see FIG. 1). Thus, the Δ4-desaturase is utilized in the last step of the method in order to create the “final” desired product. As another example, one may expose linoleic acid to a Δ6-desaturase in order to create γ-linolenic acid (GLA), and then expose the GLA to a Δ5-desaturase in order to create arachidonic acid (AA). The AA may then be exposed to an elongase in order to convert it to adrenic acid. Finally, the adrenic acid may be exposed to Δ4-desaturase in order to convert it to ω6-docosapentaenoic acid (see FIG. 1). Thus, the method involves the utilization of a linoleic acid substrate and a series of desaturase and elongase enzymes, in addition to the Δ4-desaturase, in order to arrive at the final product. By use of a similar method, one may also convert the substrate PUFA, α-linolenic acid to docosoahexaenoic acid. Again, various desaturases and elongase are used to ultimately arrive at ω3-docosapentaenoic acid which is then exposed to Δ4-desaturase (SEQ ID NO: 18, SEQ ID NO: 19, SEQ ID NO: 20 or SEQ ID NO: 21) in order to convert it to docosahexaenoic acid. (Possible substrates include those shown in FIG. 1, for example, linoleic acid, α-linolenic acid, stearidonic acid, arachidonic acid, dihomo-γ-linolenic acid, ω6-docosapentaenoic acid and eicosapentaenoic acid.)

Additionally, the present invention includes a method for producing a polyunsaturated fatty acid comprising the steps of: a) isolating the nucleotide sequence represented by SEQ ID NO: 14, SEQ ID NO: 15, SEQ ID NO: 16 or SEQ ID NO: 17; b) constructing a vector comprising the isolated nucleotide sequence; c) introducing the vector into a host cell under time and conditions sufficient for expression of Δ4-desaturase enzyme; and d) exposing the expressed Δ4-desaturase enzyme to a substrate polyunsaturated fatty acid in order to convert the substrate to a product polyunsaturated fatty acid. The substrate polyunsaturated fatty acid may be, for example, adrenic acid or ω6-docosapentaenoic acid, and the product polyunsaturated fatty acid may be, for example, ω6-docosapentaenoic acid or docosahexaenoic acid, respectively. This method may further comprise the step of exposing the product polyunsaturated fatty acid to a desaturase (or elongase) in order to convert the product polyunsaturated fatty acid to another polyunsaturated fatty acid. In this method containing the additional step, the product polyunsaturated fatty acid may be, for example, ω6-docosapentaenoic acid, and the “another” polyunsaturated fatty acid may be, for example, docosahexaenoic acid.

The present invention also encompasses a nutritional composition comprising at least one polyunsaturated fatty acid selected from the group consisting of the “product” polyunsaturated fatty acid produced according to the methods described above and the “another” polyunsaturated fatty acid produced according to the methods described above. The product polyunsaturated fatty acid may be, for example, ω6-docosapentaenoic acid or docosahexaenoic acid. The another polyunsaturated fatty acid may be, for example, docosahexaenoic acid.

The present invention also includes a pharmaceutical composition comprising 1) at least one PUFA selected from the group consisting of the product PUFA produced according to the methods described above and the another PUFA produced according to the methods described above and 2) a pharmaceutically acceptable carrier.

Additionally, the present invention encompasses an animal feed or cosmetic comprising at least one PUFA selected from the group consisting of the product PUFA produced according to the methods described above and the another PUFA produced according to the methods described. These PUFAs have been listed above and are exemplified in FIG. 1.

Additionally, the present invention encompasses a method of preventing or treating a condition caused by insufficient intake of polyunsaturated fatty acids comprising administering to the patient the nutritional composition above in an amount sufficient to effect prevention or treatment.

### BRIEF DESCRIPTION OF THE DRAWINGS

FIG. 1 illustrates the fatty acid biosynthetic pathway and the role of Δ4-desaturase in this pathway.

FIG. 2 illustrates an amino acid comparison of Δ4-desaturases produced by four different plasmids (SEQ ID NO: 18, SEQ ID NO: 19, SEQ ID NO: 20 and SEQ ID NO: 21).

FIG. 3 illustrates the nucleotide sequence encoding Δ4-desaturase of Thraustochytrium aureum (ATCC 34304) from plasmid pRTA5 (SEQ ID NO: 14).

FIG. 4 illustrates the nucleotide sequence encoding Δ4-desaturase of Thraustochytrium aureum (ATCC 34304) from plasmid pRTA6 (SEQ ID NO: 15).

FIG. 5 illustrates the nucleotide sequence encoding Δ4-desaturase of Thraustochytrium aureum (ATCC 34304) from plasmid pRTA7 (SEQ ID NO: 16).

FIG. 6 illustrates the nucleotide sequence encoding encoding Δ4-desaturase of Thraustochytrium aureum (ATCC 34304) from plasmid pRTA8 (SEQ ID NO: 17).

FIG. 7 illustrates the amino acid sequence of Δ4-desaturase of Thraustochytrium aureum (ATCC 34304) from plasmid pRTA5 (SEQ ID NO: 18).

FIG. 8 illustrates the amino acid sequence of Δ4-desaturase of Thraustochytrium aureum (ATCC 34304) from plasmid pRTA6 (SEQ ID NO: 19).

FIG. 9 illustrates the amino acid sequence of Δ4-desaturase of Thraustochytrium aureum (ATCC 34304) from plasmid pRTA7 (SEQ ID NO: 20).

FIG. 10 illustrates the amino acid sequence of Δ4-desaturase of Thraustochytrium aureum (ATCC 34304) from plasmid pRTA8 (SEQ ID NO: 21).

FIG. 11 illustrates the nucleotide and amino acid sequences described herein.

FIG. 12 illustrates the DNA sequence of the elongase gene MELO4 from a mouse.

FIG. 13 illustrates the DNA sequence of the putatative Δ4-desaturase ssa.con (SEQ ID NO: 24) generated from clones saa9 and saa5 from S. aggregatum (ATCC 28209) (see Example VI).

FIG. 14 illustrates the amino acid sequence (SEQ ID NO: 25) of the putative Δ4-desaturase encoded by the ssa.con DNA sequence from S. aggregatum (ATCC 28209) (see Example VI).

FIG. 15 illustrates the alignment of the amino acids derived from the translation of the open reading frames of ssa.con DNA from S. aggregatum (ATCC 28209) and pRTA7 (see Example VI).

### DETAILED DESCRIPTION OF THE INVENTION

The subject invention relates to the nucleotide and translated amino acid sequences of the Δ4-desaturase gene derived from the fungus Thraustochytrium aureum (ATCC 34304). Furthermore, the subject invention also includes uses of this gene and of the enzyme encoded by this gene. For example, the gene and corresponding enzyme may be used in the production of polyunsaturated fatty acids such as, for instance, ω6-docosapentaenoic acid and/or docosahexaenoic acid which may be added to pharmaceutical compositions, nutritional compositions and to other valuable products.

The Δ4-Desaturase Gene and Enzyme Encoded Thereby

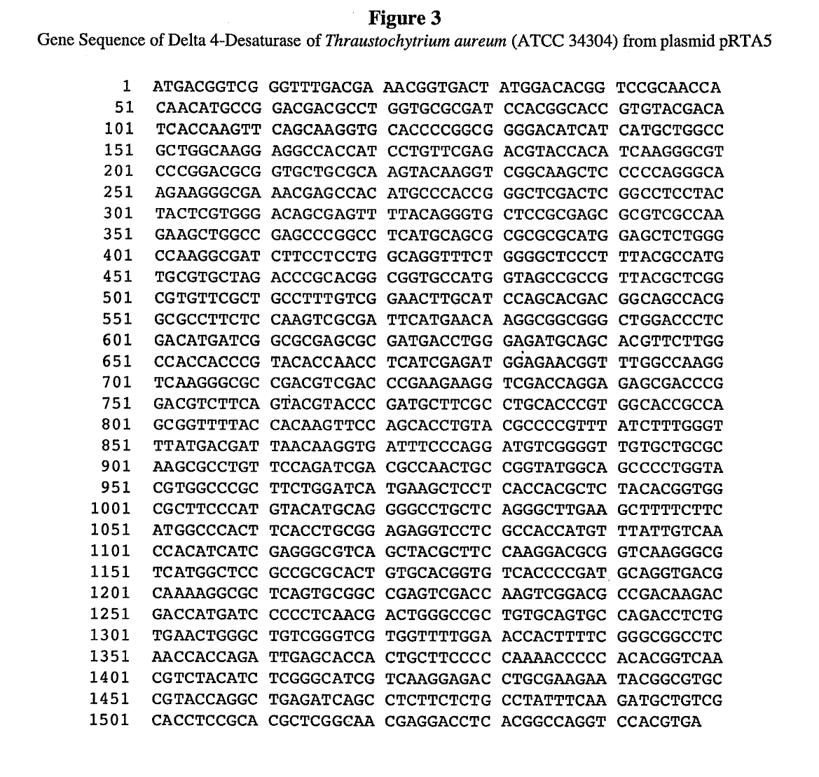

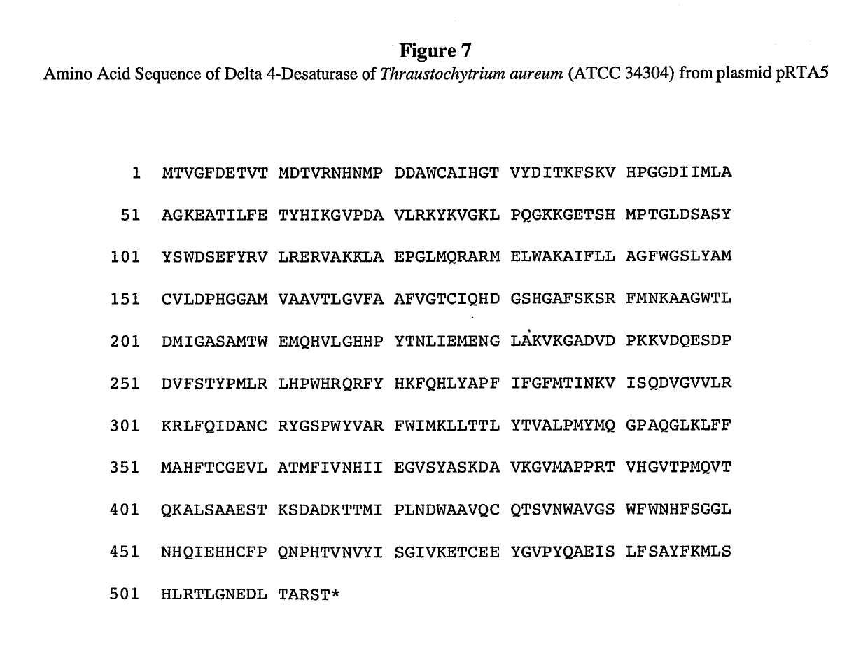

As noted above, the enzyme encoded by the Δ4-desaturase gene of the present invention is essential in the production of highly unsaturated polyunsaturated fatty acids having a length greater than 22 carbons. The nucleotide sequences of the isolated Thraustochytrium aureum Δ4-desaturase gene, which differed based upon the plasmid created (see Example III), are shown in FIGS. 3-6, and the amino acid sequences of the corresponding purified proteins are shown in FIG. 7-10.

As an example of the importance of the genes of the present invention, the isolated Δ4-desaturase genes convert adrenic acid to ω6-docosapentaenoic acid or convert ω3-docosapentaenoic acid to docosahexaenoic acid.

It should be noted that the present invention also encompasses nucleotide sequences (and the corresponding encoded proteins) having sequences corresponding to, identical to, or complementary to at least about 50%, preferably at least about 60%, and more preferably at least about 70% of the nucleotides in sequence to SEQ ID NO: 14, SEQ ID NO: 15, SEQ ID NO: 16 or SEQ ID NO: 17 (i.e., the nucleotide sequences of the Δ4-desaturase gene of Thraustochytrium aureum) described herein. Such sequences may be derived from fungal sources as well as other non-fungal sources (e.g., C. elegans, mouse or human). Furthermore, the present invention also encompasses fragments and derivatives of the nucleotide sequences of the present invention (i.e., SEQ ID NO: 14, SEQ ID NO: 15, SEQ ID NO: 16 or SEQ ID NO: 17), as well as of the sequences derived from other sources, and having the above-described complementarity, identity or correspondence. Functional equivalents of the above-sequences (i.e., sequences having Δ4-desaturase activity, as appropriate) are also encompassed by the present invention.

For purposes of the present invention, a “fragment” is of a nucleotide sequence is defined as a contiguous sequence of approximately at least 6, preferably at least about 8, more preferably at least about 10-12 nucleotides, and even more preferably at least about 15-18 nucleotides corresponding to a region of the specified nucleotide sequence.

Furthermore, for purposes of the present invention, a “complement” is defined as a sequence which pairs to a given sequence based upon base-pairing rules. For example, a sequence A-G-T in one nucleotide strand is “complementary” to T-C-A in the other strand.

Sequence identity or percent identity is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm may be extended to use with peptide or protein sequences using the scoring matrix created by Dayhoff, Atlas of Protein Sequences and Structure, M. O. Dayhoff ed., 5 Suppl. 3:353-358, National Biomedical Research Foundation, Washington, D.C., USA, and normalized by Gribskov, Nucl. Acids Res. 14(6):6745-66763 (1986). An implementation of this algorithm for nucleic acid and peptide sequences is provided by the Genetics Computer Group (Madison, Wis.) in the BestFit utility application. The default parameters for this method are described in the Wisconsin Sequence Analysis Package Program Manual, Version 8 (1995) (available from Genetics Computer Group, Madison, Wis.). Other equally suitable programs for calculating the percent identity or similarity between sequences are generally known in the art.

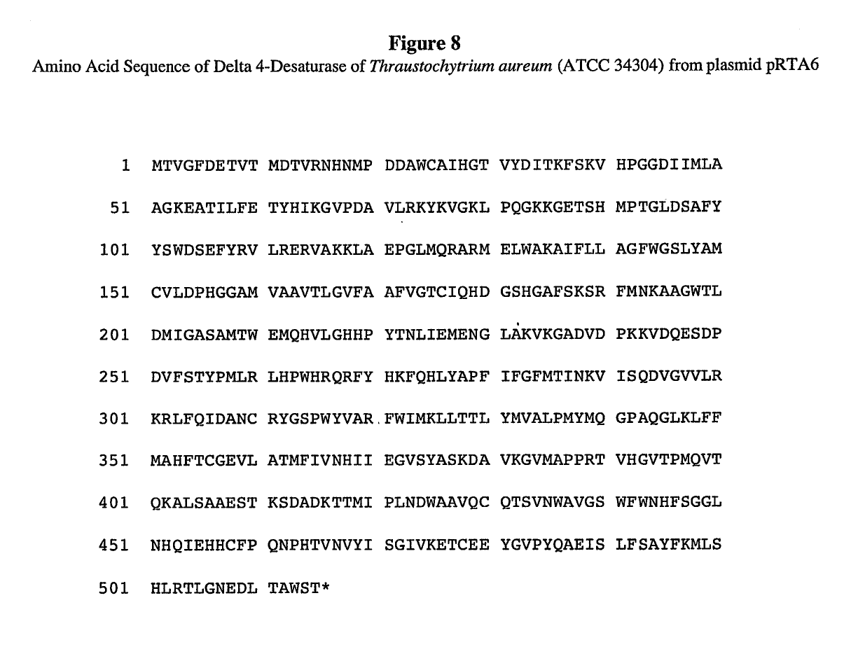

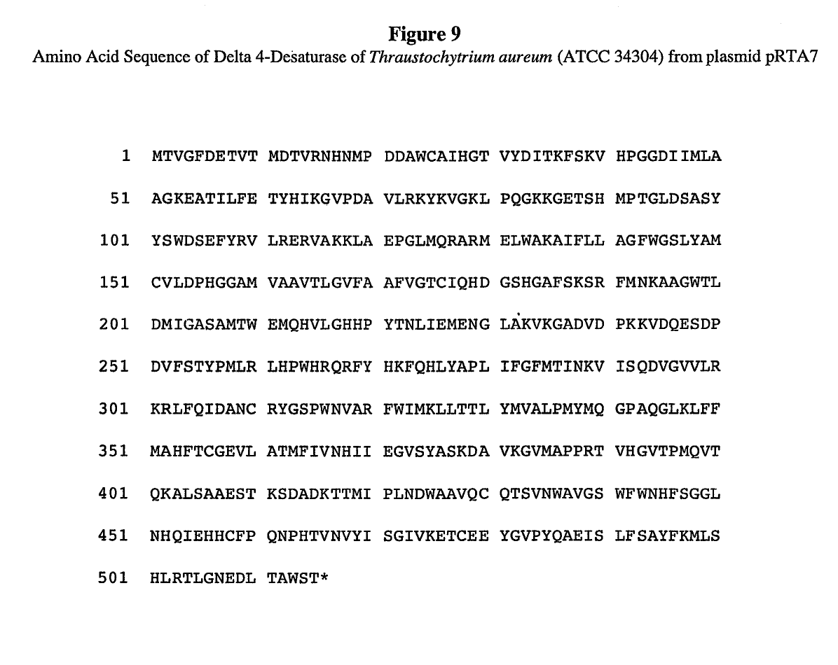

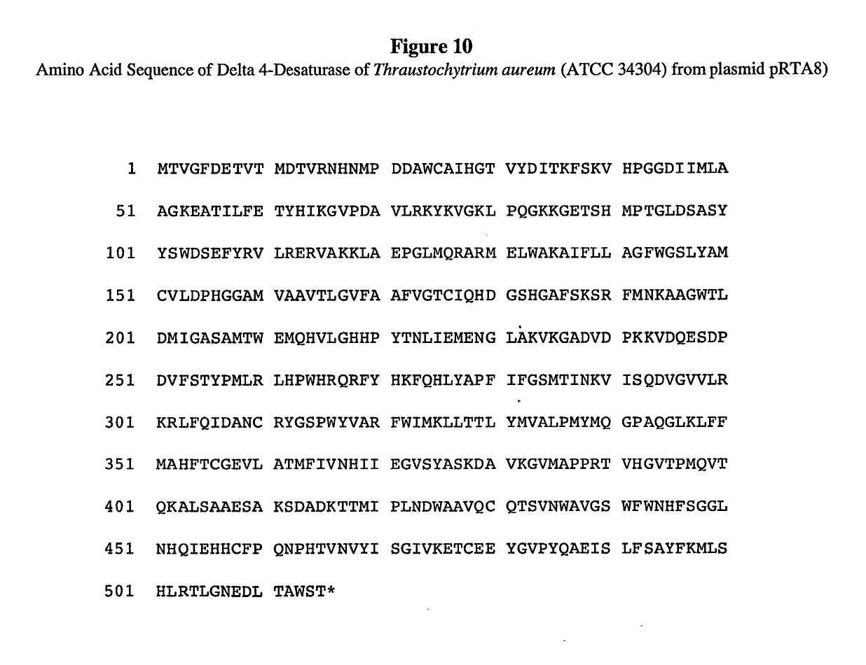

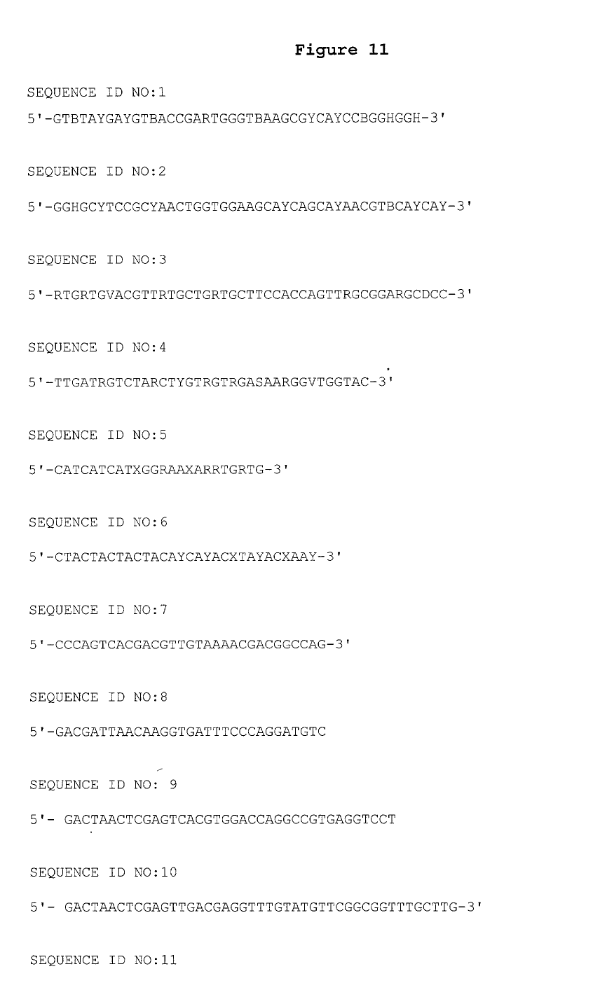

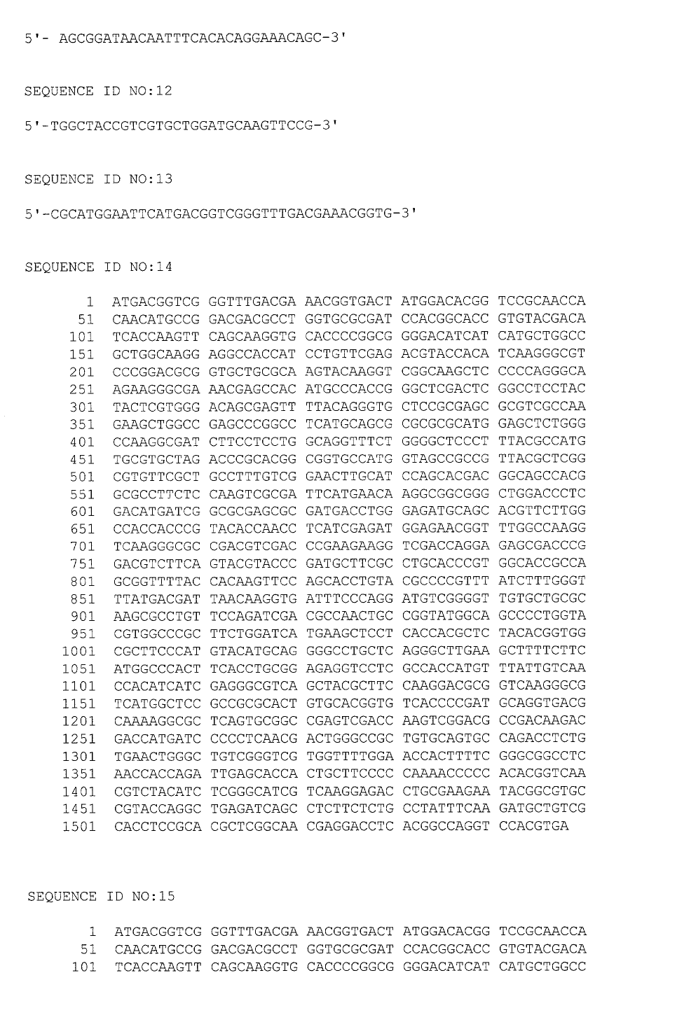

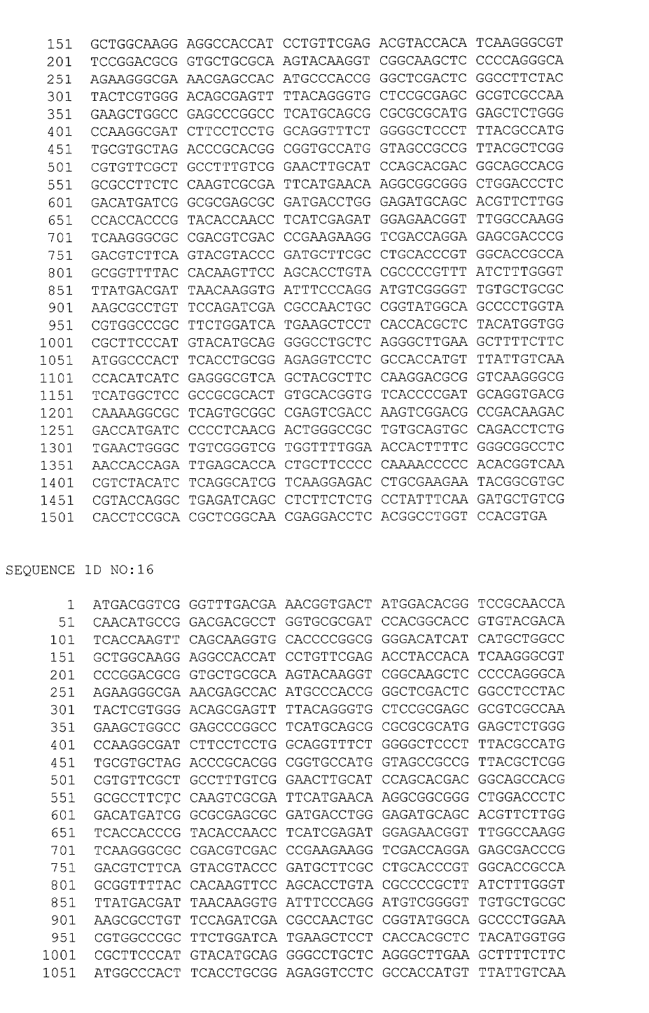

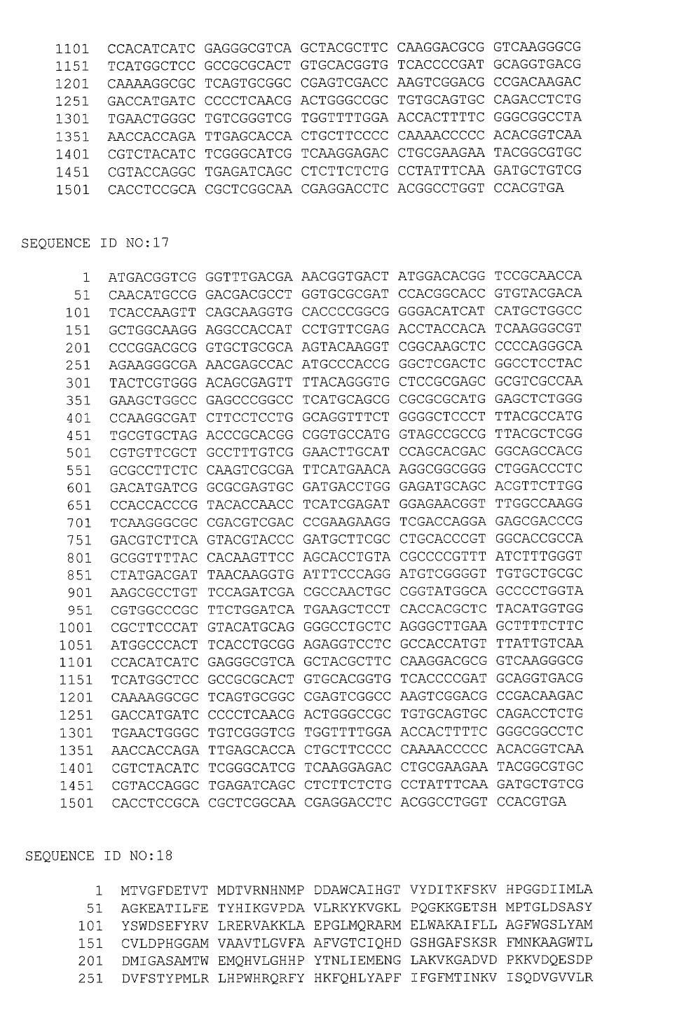

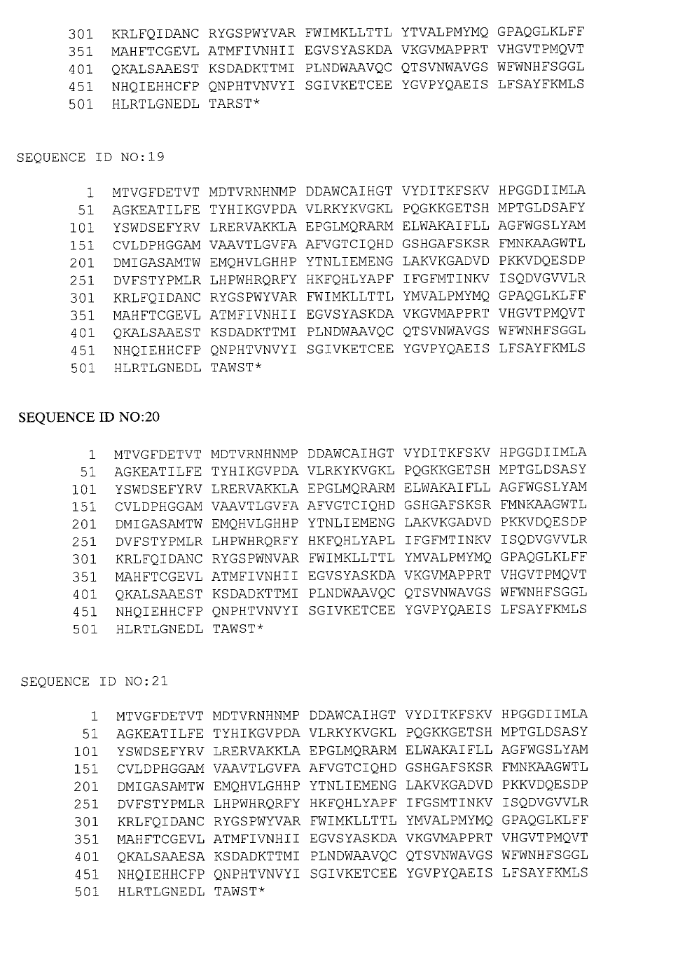

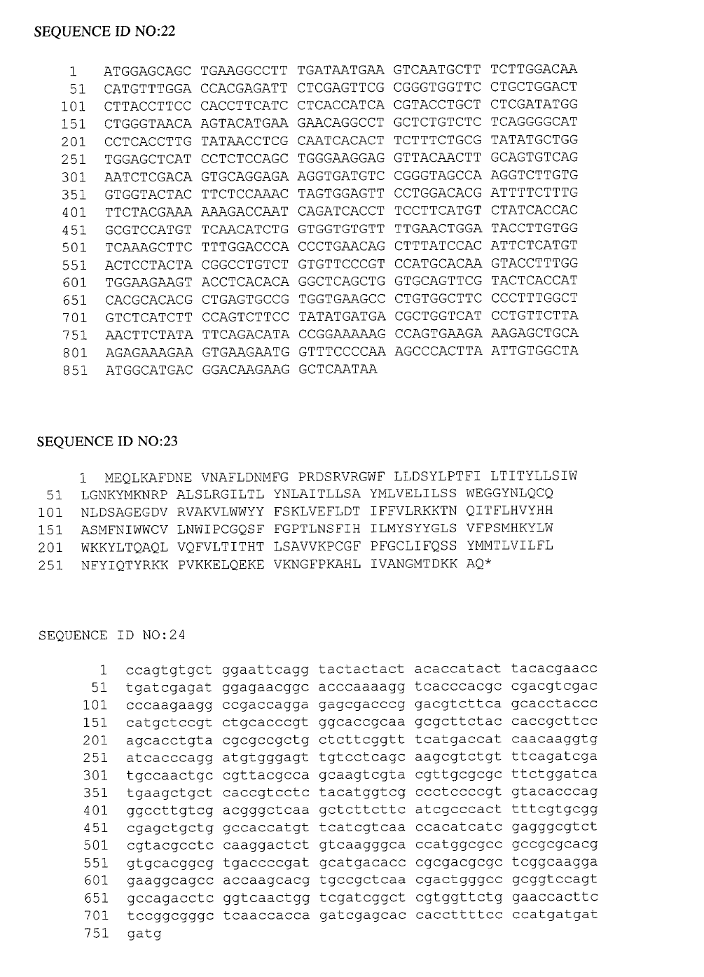

The invention also includes a purified polypeptide which desaturates polyunsaturated fatty acids at the carbon 4 position and has at least about 50% amino acid similarity, preferably at least about 60% similarity, and more preferably at least about 70% similarity to the amino acid sequences (i.e., SEQ ID NO: 18 (shown in FIG. 7), SEQ ID NO: 19 (shown in FIG. 8), SEQ ID NO: 20 (shown in FIG. 9) and SEQ ID NO: 21 (shown in FIG. 10)) of the above-noted proteins which are, in turn, encoded by the above-described nucleotide sequences.

For purposes of the present invention, “similarity” is defined as the exact amino acid to amino acid comparison of two or more polypeptides at the appropriate place, where amino acids are identical or possess similar chemical and/or physical properties such as charge or hydrophobicity. “Percent similarity” is calculated between the compared polypeptide sequences using programs known in the art (see above).

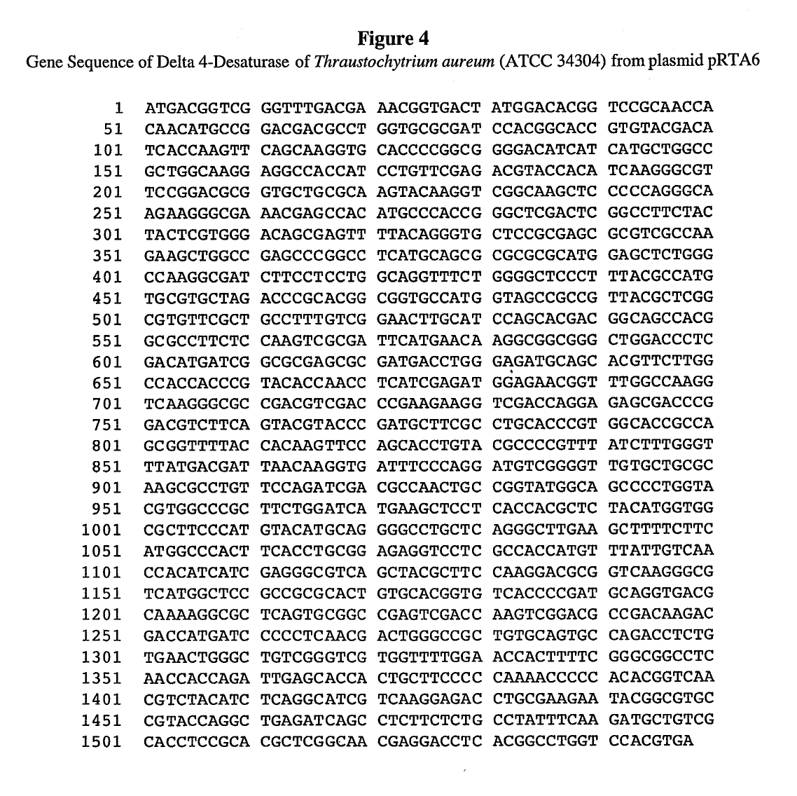

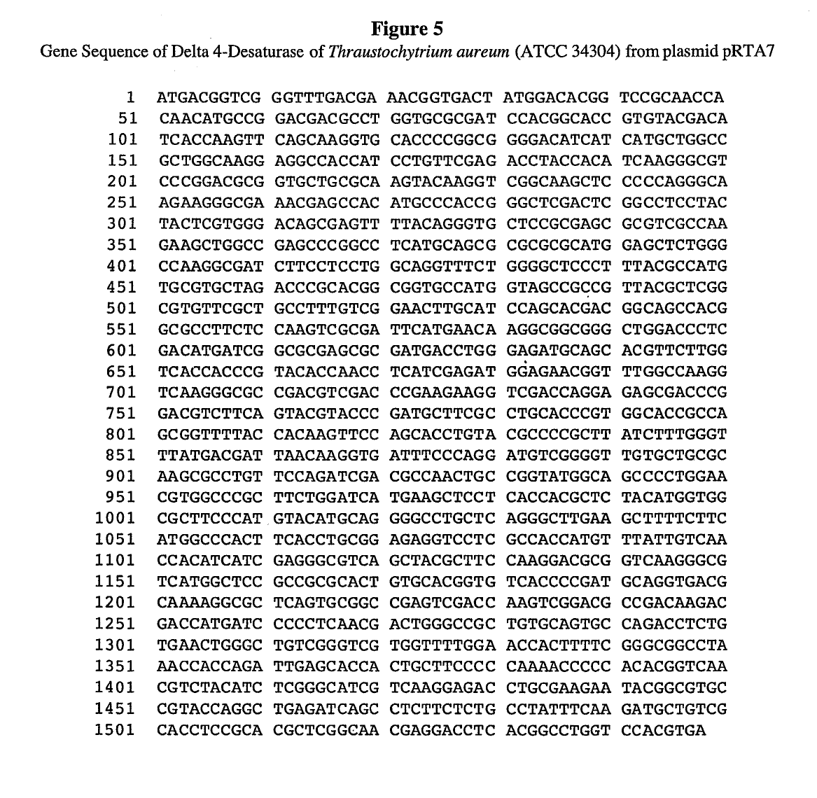

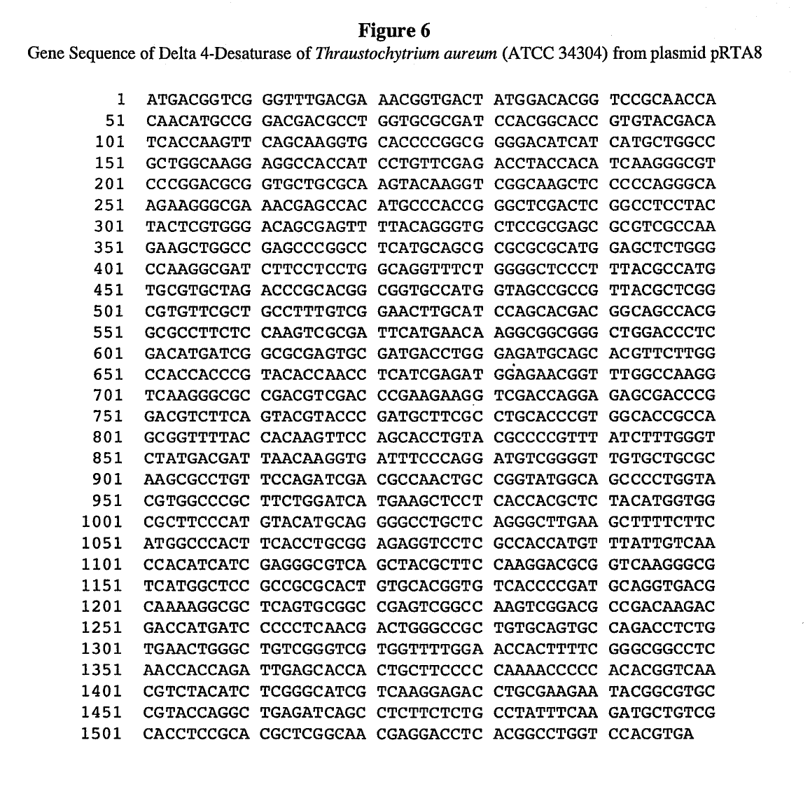

The present invention also encompasses an isolated nucleotide sequence which encodes PUFA desaturase activity and that is hybridizable, under moderately stringent conditions, to a nucleic acid having a nucleotide sequence corresponding to or complementary to the nucleotide sequence comprising or represented by SEQ ID NO: 14 (shown in FIG. 3), SEQ ID NO: 15 (shown in FIG. 4), SEQ ID NO: 16 (shown in FIG. 5), or SEQ ID NO: 17 (shown in FIG. 6). A nucleic acid molecule is “hybridizable” to another nucleic acid molecule when a single-stranded form of the nucleic acid molecule can anneal to the other nucleic acid molecule under the appropriate conditions of temperature and ionic strength (see Sambrook et al., “Molecular Cloning: A Laboratory Manual, Second Edition (1989), Cold Spring Harbor Laboratory Press, Cold Spring Harbor, N.Y.)). The conditions of temperature and ionic strength determine the “stringency” of the hybridization. “Hybridization” requires that two nucleic acids contain complementary sequences. However, depending on the stringency of the hybridization, mismatches between bases may occur. The appropriate stringency for hybridizing nucleic acids depends on the length of the nucleic acids and the degree of complementation. Such variables are well known in the art. More specifically, the greater the degree of similarity or homology between two nucleotide sequences, the greater the value of Tm for hybrids of nucleic acids having those sequences. For hybrids of greater than 100 nucleotides in length, equations for calculating Tm have been derived (see Sambrook et al., supra). For hybridization with shorter nucleic acids, the position of mismatches becomes more important, and the length of the oligonucleotide determines its specificity (see Sambrook et al., supra).

Production of the Δ4-Desaturase Enyzme

Once the gene encoding the Δ4-desaturase enzyme has been isolated, it may then be introduced into either a prokaryotic or eukaryotic host cell through the use of a vector or construct. The vector, for example, a bacteriophage, cosmid or plasmid, may comprise the nucleotide sequence encoding the Δ4-desaturase enzyme, as well as any promoter which is functional in the host cell and is able to elicit expression of the desaturase encoded by the nucleotide sequence. The promoter is in operable association with or operably linked to the nucleotide sequence. (A promoter is said to be “operably linked” with a coding sequence if the promoter affects transcription or expression of the coding sequence.) Suitable promoters include, for example, those from genes encoding alcohol dehydrogenase, glyceraldehyde-3-phosphate dehydrogenase, phosphoglucoisomerase, phosphoglycerate kinase, acid phosphatase, T7, TPI, lactase, metallothionein, cytomegalovirus immediate early, whey acidic protein, glucoamylase, and promoters activated in the presence of galactose, for example, GAL1 and GAL10. Additionally, nucleotide sequences which encode other proteins, oligosaccharides, lipids, etc. may also be included within the vector as well as other regulatory sequences such as a polyadenylation signal (e.g., the poly-A signal of SV-40T-antigen, ovalalbumin or bovine growth hormone). The choice of sequences present in the construct is dependent upon the desired expression products as well as the nature of the host cell.

As noted above, once the vector has been constructed, it may then be introduced into the host cell of choice by methods known to those of ordinary skill in the art including, for example, transfection, transformation and electroporation (see Molecular Cloning: A Laboratory Manual, 2^{nd} ed., Vol. 1-3, ed. Sambrook et al., Cold Spring Harbor Laboratory Press (1989)). The host cell is then cultured under suitable conditions permitting expression of the genes leading to the production of the desired PUFA, which is then recovered and purified.

Examples of suitable prokaryotic host cells include, for example, bacteria such as Escherichia coli, Bacillus subtilis as well as cyanobacteria such as Spirulina spp. (i.e., blue-green algae). Examples of suitable eukaryotic host cells include, for example, mammalian cells, plant cells, yeast cells such as Saccharomyces cerevisiae, Saccharomyces carlsbergensis, Lipomyces starkey, Candida spp. such as Yarrowia (Candida) lipolytica, Kluyveromyces spp., Pichia spp., Trichoderma spp. or Hansenula spp., or fungal cells such as filamentous fungal cells, for example, Aspergillus, Neurospora and Penicillium. Preferably, Saccharomyces cerevisiae (baker's yeast) cells are utilized.

Expression in a host cell can be accomplished in a transient or stable fashion. Transient expression can occur from introduced constructs which contain expression signals functional in the host cell, but which constructs do not replicate and rarely integrate in the host cell, or where the host cell is not proliferating. Transient expression also can be accomplished by inducing the activity of a regulatable promoter operably linked to the gene of interest, although such inducible systems frequently exhibit a low basal level of expression. Stable expression can be achieved by introduction of a construct that can integrate into the host genome or that autonomously replicates in the host cell. Stable expression of the gene of interest can be selected through the use of a selectable marker located on or transfected with the expression construct, followed by selection for cells expressing the marker. When stable expression results from integration, the site of the construct's integration can occur randomly within the host genome or can be targeted through the use of constructs containing regions of homology with the host genome sufficient to target recombination with the host locus. Where constructs are targeted to an endogenous locus, all or some of the transcriptional and translational regulatory regions can be provided by the endogenous locus.

A transgenic mammal may also be used in order to express the enzyme of interest (i.e., Δ4-desaturase), and ultimately the PUFA(s) of interest. More specifically, once the above-described construct is created, it may be inserted into the pronucleus of an embryo. The embryo may then be implanted into a recipient female. Alternatively, a nuclear transfer method could also be utilized (Schnieke et al., Science 278:2130-2133 (1997)). Gestation and birth are then permitted (see, e.g., U.S. Pat. No. 5,750,176 and U.S. Pat. No. 5,700,671). Milk, tissue or other fluid samples from the offspring should then contain altered levels of PUFAs, as compared to the levels normally found in the non-transgenic animal. Subsequent generations may be monitored for production of the altered or enhanced levels of PUFAs and thus incorporation of the gene encoding the desired desaturase enzyme into their genomes. The mammal utilized as the host may be selected from the group consisting of, for example, a mouse, a rat, a rabbit, a pig, a goat, a sheep, a horse and a cow. However, any mammal may be used provided it has the ability to incorporate DNA encoding the enzyme of interest into its genome.

For expression of a desaturase polypeptide, functional transcriptional and translational initiation and termination regions are operably linked to the DNA encoding the desaturase polypeptide. Transcriptional and translational initiation and termination regions are derived from a variety of nonexclusive sources, including the DNA to be expressed, genes known or suspected to be capable of expression in the desired system, expression vectors, chemical synthesis, or from an endogenous locus in a host cell. Expression in a plant tissue and/or plant part presents certain efficiencies, particularly where the tissue or part is one which is harvested early, such as seed, leaves, fruits, flowers, roots, etc. Expression can be targeted to that location with the plant by utilizing specific regulatory sequence such as those of U.S. Pat. Nos. 5,463,174, 4,943,674, 5,106,739, 5,175,095, 5,420,034, 5,188,958, and 5,589,379. Alternatively, the expressed protein can be an enzyme which produces a product which may be incorporated, either directly or upon further modifications, into a fluid fraction from the host plant. Expression of a desaturase gene, or antisense desaturase transcripts, can alter the levels of specific PUFAs, or derivatives thereof, found in plant parts and/or plant tissues. The desaturase polypeptide coding region may be expressed either by itself or with other genes, in order to produce tissues and/or plant parts containing higher proportions of desired PUFAs or in which the PUFA composition more closely resembles that of human breast milk (Prieto et al., PCT publication WO 95/24494). The termination region may be derived from the 3′ region of the gene from which the initiation region was obtained or from a different gene. A large number of termination regions are known to and have been found to be satisfactory in a variety of hosts from the same and different genera and species. The termination region usually is selected as a matter of convenience rather than because of any particular property.

As noted above, a plant (e.g., Glycine max (soybean) or Brassica napus (canola)) or plant tissue may also be utilized as a host or host cell, respectively, for expression of the desaturase enzyme which may, in turn, be utilized in the production of polyunsaturated fatty acids. More specifically, desired PUFAS can be expressed in seed. Methods of isolating seed oils are known in the art. Thus, in addition to providing a source for PUFAs, seed oil components may be manipulated through the expression of the desaturase gene, as well as perhaps other desaturase genes and elongase genes, in order to provide seed oils that can be added to nutritional compositions, pharmaceutical compositions, animal feeds and cosmetics. Once again, a vector which comprises a DNA sequence encoding the desaturase operably linked to a promoter, will be introduced into the plant tissue or plant for a time and under conditions sufficient for expression of the desaturase gene. The vector may also comprise one or more genes that encode other enzymes, for example, Δ5-desaturase, elongase, Δ12-desaturase, Δ15-desaturase, Δ17-desaturase, and/or Δ19-desaturase. The plant tissue or plant may produce the relevant substrate (e.g., adrenic acid or ω3-docosapentaenoic acid) upon which the enzyme acts or a vector encoding enzymes which produce such substrates may be introduced into the plant tissue, plant cell or plant. In addition, substrate may be sprayed on plant tissues expressing the appropriate enzymes. Using these various techniques, one may produce PUFAs (e.g., n-6 unsaturated fatty acids such as ω6-docosapentaenoic acid, or n-3 fatty acids such as docosahexaenoic acid) by use of a plant cell, plant tissue or plant. It should also be noted that the invention also encompasses a transgenic plant comprising the above-described vector, wherein expression of the nucleotide sequence of the vector results in production of a polyunsaturated fatty acid in, for example, the seeds of the transgenic plant.

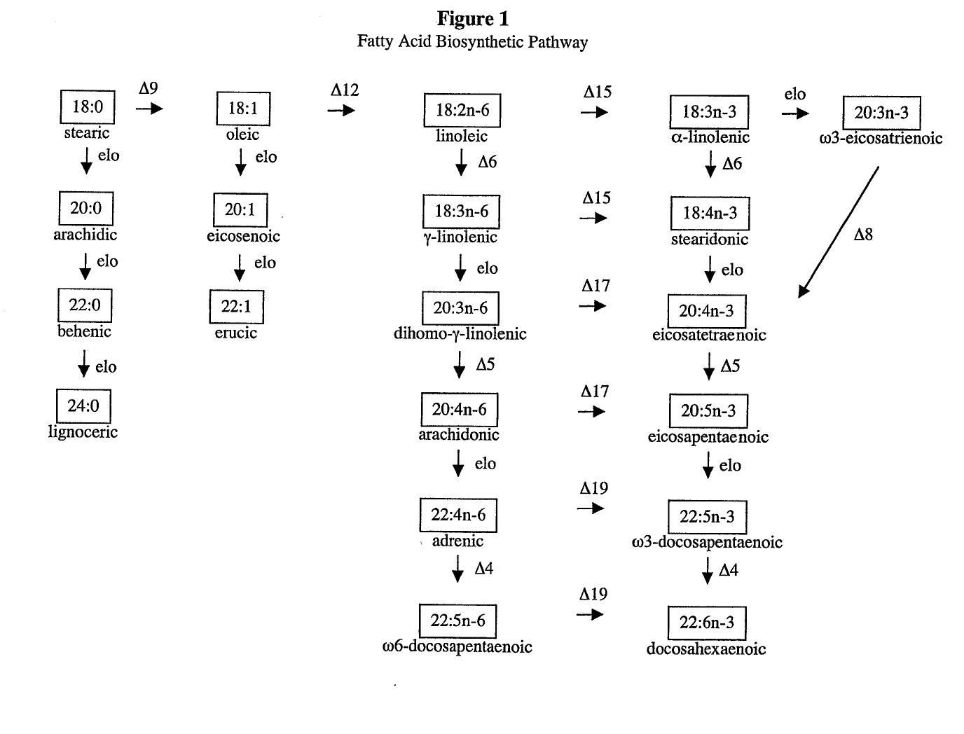

The substrates which may be produced by the host cell either naturally or transgenically, as well as the enzymes which may be encoded by DNA sequences present in the vector which is subsequently introduced into the host cell, are shown in FIG. 1.

In view of the above, the present invention encompasses a method of producing the Δ4-desaturase enzyme comprising the steps of: 1) isolating the nucleotide sequence of the gene encoding the desaturase enzyme; 2) constructing a vector comprising said nucleotide sequence; and 3) introducing said vector into a host cell under time and conditions sufficient for the production of the desaturase enzyme.

The present invention also encompasses a method of producing polyunsaturated fatty acids comprising exposing an acid to the enzyme such that the desaturase converts the acid to a polyunsaturated fatty acid. For example, when 22:4n-6 is exposed to the Δ4-desaturase enzyme, it is converted to ω6-docosapentaenoic acid. This acid may then be exposed to Δ19-desaturase which converts the acid to docosahexaenoic acid.

Uses of the Δ4-Desaturase Gene and Enzyme Encoded Thereby

As noted above, the isolated desaturase genes and the desaturase enzyme encoded thereby have many uses. For example, the gene and corresponding enzyme may be used indirectly or directly in the production of polyunsaturated fatty acids, for example, Δ4-desaturase may be used in the production of ω6-docosapentaenoic acid or docosahexaenoic acid. (“Directly” is meant to encompass the situation where the enzyme directly converts the acid to another acid, the latter of which is utilized in a composition (e.g., the conversion of adrenic acid to ω6-docosapentaenoic acid). “Indirectly” is meant to encompass the situation where an acid is converted to another acid (i.e., a pathway intermediate) by the desaturase (e.g., adrenic acid to ω6-docosapentaenoic acid) and then the latter acid is converted to another acid by use of a desaturase or non-desaturase enzyme (e.g., ω6-docosapentaenoic acid to docosahexaenoic acid by Δ19-desaturase). These polyunsaturated fatty acids (i.e., those produced either directly or indirectly by activity of the desaturase enzyme) may be added to, for example, nutritional compositions, pharmaceutical compositions, cosmetics, and animal feeds, all of which are encompassed by the present invention. These uses are described, in detail, below.

Nutritional Compositions

The present invention includes nutritional compositions. Such compositions, for purposes of the present invention, include any food or preparation for human consumption including for enteral or parenteral consumption, which when taken into the body (a) serve to nourish or build up tissues or supply energy and/or (b) maintain, restore or support adequate nutritional status or metabolic function.

The nutritional composition of the present invention comprises at least one oil or acid produced directly or indirectly by use of the desaturase gene, in accordance with the present invention, and may either be in a solid or liquid form. Additionally, the composition may include edible macronutrients, vitamins and minerals in amounts desired for a particular use. The amount of such ingredients will vary depending on whether the composition is intended for use with normal, healthy infants, children or adults having specialized needs such as those which accompany certain metabolic conditions (e.g., metabolic disorders).

Examples of macronutrients which may be added to the composition include but are not limited to edible fats, carbohydrates and proteins. Examples of such edible fats include but are not limited to coconut oil, borage oil, fungal oil, black current oil, soy oil, and mono- and diglycerides. Examples of such carbohydrates include but are not limited to glucose, edible lactose and hydrolyzed search. Additionally, examples of proteins which may be utilized in the nutritional composition of the invention include but are not limited to soy proteins, electrodialysed whey, electrodialysed skim milk, milk whey, or the hydrolysates of these proteins.

With respect to vitamins and minerals, the following may be added to the nutritional compositions of the present invention: calcium, phosphorus, potassium, sodium, chloride, magnesium, manganese, iron, copper, zinc, selenium, iodine, and Vitamins A, E, D, C, and the B complex. Other such vitamins and minerals may also be added.

The components utilized in the nutritional compositions of the present invention will be of semi-purified or purified origin. By semi-purified or purified is meant a material which has been prepared by purification of a natural material or by synthesis.

Examples of nutritional compositions of the present invention include but are not limited to infant formulas, dietary supplements, dietary substitutes, and rehydration compositions. Nutritional compositions of particular interest include but are not limited to those utilized for enteral and parenteral supplementation for infants, specialist infant formulas, supplements for the elderly, and supplements for those with gastrointestinal difficulties and/or malabsorption.

The nutritional composition of the present invention may also be added to food even when supplementation of the diet is not required. For example, the composition may be added to food of any type including but not limited to margarines, modified butters, cheeses, milk, yogurt, chocolate, candy, snacks, salad oils, cooking oils, cooking fats, meats, fish and beverages.

In a preferred embodiment of the present invention, the nutritional composition is an enteral nutritional product, more preferably, an adult or pediatric enteral nutritional product. This composition may be administered to adults or children experiencing stress or having specialized needs due to chronic or acute disease states. The composition may comprise, in addition to polyunsaturated fatty acids produced in accordance with the present invention, macronutrients, vitamins and minerals as described above. The macronutrients may be present in amounts equivalent to those present in human milk or on an energy basis, i.e., on a per calorie basis.

Methods for formulating liquid or solid enteral and parenteral nutritional formulas are well known in the art. (See also the Examples below.)

The enteral formula, for example, may be sterilized and subsequently utilized on a ready-to-feed (RTF) basis or stored in a concentrated liquid or powder. The powder can be prepared by spray drying the formula prepared as indicated above, and reconstituting it by rehydrating the concentrate. Adult and pediatric nutritional formulas are well known in the art and are commercially available (e.g., Similac®, Ensure®, Jevity® and Alimentum® from Ross Products Division, Abbott Laboratories, Columbus, Ohio). An oil or acid produced in accordance with the present invention may be added to any of these formulas.

The energy density of the nutritional compositions of the present invention, when in liquid form, may range from about 0.6 Kcal to about 3 Kcal per ml. When in solid or powdered form, the nutritional supplements may contain from about 1.2 to more than 9 Kcals per gram, preferably about 3 to 7 Kcals per gm. In general, the osmolality of a liquid product should be less than 700 mOsm and, more preferably, less than 660 mOsm.

The nutritional formula may include macronutrients, vitamins, and minerals, as noted above, in addition to the PUFAs produced in accordance with the present invention. The presence of these additional components helps the individual ingest the minimum daily requirements of these elements. In addition to the provision of PUFAs, it may also be desirable to add zinc, copper, folic acid and antioxidants to the composition. It is believed that these substances boost a stressed immune system and will therefore provide further benefits to the individual receiving the composition. A pharmaceutical composition may also be supplemented with these elements.

In a more preferred embodiment, the nutritional composition comprises, in addition to antioxidants and at least one PUFA, a source of carbohydrate wherein at least 5 weight percent of the carbohydrate is indigestible oligosaccharide. In a more preferred embodiment, the nutritional composition additionally comprises protein, taurine, and carnitine.

As noted above, the PUFAs produced in accordance with the present invention, or derivatives thereof, may be added to a dietary substitute or supplement, particularly an infant formula, for patients undergoing intravenous feeding or for preventing or treating malnutrition or other conditions or disease states. As background, it should be noted that human breast milk has a fatty acid profile comprising from about 0.15% to about 0.36% as DHA, from about 0.03% to about 0.13% as EPA, from about 0.30% to about 0.88% as AA, from about 0.22% to about 0.67% as DGLA, and from about 0.27% to about 1.04% as GLA. Thus, fatty acids such as AA, EPA and/or docosahexaenoic acid (DHA), produced in accordance with the present invention, can be used to alter, for example, the composition of infant formulas in order to better replicate the PUFA content of human breast milk or to alter the presence of PUFAs normally found in a non-human mammal's milk. In particular, a composition for use in a pharmacologic or food supplement, particularly a breast milk substitute or supplement, will preferably comprise one or more of AA, DGLA and GLA. More preferably, the oil will comprise from about 0.3 to 30% AA, from about 0.2 to 30% DGLA, and/or from about 0.2 to about 30% GLA.

Parenteral nutritional compositions comprising from about 2 to about 30 weight percent fatty acids calculated as triglycerides are encompassed by the present invention. The preferred composition has about 1 to about 25 weight percent of the total PUFA composition as GLA (U.S. Pat. No. 5,196,198). Other vitamins, particularly fat-soluble vitamins such as vitamin A, D, E and L-carnitine can optionally be included. When desired, a preservative such as alpha-tocopherol may be added in an amount of about 0.1% by weight.

In addition, the ratios of AA, DGLA and GLA can be adapted for a particular given end use. When formulated as a breast milk supplement or substitute, a composition which comprises one or more of AA, DGLA and GLA will be provided in a ratio of about 1:19:30 to about 6:1:0.2, respectively. For example, the breast milk of animals can vary in ratios of AA:DGLA:GLA ranging from 1:19:30 to 6:1:0.2, which includes intermediate ratios which are preferably about 1:1:1, 1:2:1, 1:1:4. When produced together in a host cell, adjusting the rate and percent of conversion of a precursor substrate such as GLA and DGLA to AA can be used to precisely control the PUFA ratios. For example, a 5% to 10% conversion rate of DGLA to AA can be used to produce an AA to DGLA ratio of about 1:19, whereas a conversion rate of about 75% TO 80% can be used to produce an AA to DGLA ratio of about 6:1. Therefore, whether in a cell culture system or in a host animal, regulating the timing, extent and specificity of desaturase expression, as well as the expression of other desaturases and elongases, can be used to modulate PUFA levels and ratios. The PUFAs produced in accordance with the present invention (e.g., AA and EPA) may then be combined with other PUFAs/acids (e.g., GLA) in the desired concentrations and ratios.

Additionally, PUFA produced in accordance with the present invention or host cells containing them may also be used as animal food supplements to alter an animal's tissue or milk fatty acid composition to one more desirable for human or animal consumption.

Pharmaceutical Compositions

The present invention also encompasses a pharmaceutical composition comprising one or more of the acids and/or resulting oils produced using the desaturase genes, in accordance with the methods described herein. More specifically, such a pharmaceutical composition may comprise one or more of the acids and/or oils as well as a standard, well-known, non-toxic pharmaceutically acceptable carrier, adjuvant or vehicle such as, for example, phosphate buffered saline, water, ethanol, polyols, vegetable oils, a wetting agent or an emulsion such as a water/oil emulsion. The composition may be in either a liquid or solid form. For example, the composition may be in the form of a tablet, capsule, ingestible liquid or powder, injectible, or topical ointment or cream. Proper fluidity can be maintained, for example, by the maintenance of the required particle size in the case of dispersions and by the use of surfactants. It may also be desirable to include isotonic agents, for example, sugars, sodium chloride and the like. Besides such inert diluents, the composition can also include adjuvants, such as wetting agents, emulsifying and suspending agents, sweetening agents, flavoring agents and perfuming agents.

Suspensions, in addition to the active compounds, may comprise suspending agents such as, for example, ethoxylated isostearyl alcohols, polyoxyethylene sorbitol and sorbitan esters, microcrystalline cellulose, aluminum metahydroxide, bentonite, agar-agar and tragacanth or mixtures of these substances.

Solid dosage forms such as tablets and capsules can be prepared using techniques well known in the art. For example, PUFAs produced in accordance with the present invention can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and the relevant PUFA(s). The antioxidant and PUFA components should fit within the guidelines presented above.

For intravenous administration, the PUFAs produced in accordance with the present invention or derivatives thereof may be incorporated into commercial formulations such as Intralipids™. The typical normal adult plasma fatty acid profile comprises 6.64 to 9.46% of AA, 1.45 to 3.11% of DGLA, and 0.02 to 0.08% of GLA. These PUFAs or their metabolic precursors can be administered alone or in combination with other PUFAs in order to achieve a normal fatty acid profile in a patient. Where desired, the individual components of the formulations may be provided individually, in kit form, for single or multiple use. A typical dosage of a particular fatty acid is from 0.1 mg to 20 g (up to 100 g) daily and is preferably from 10 mg to 1, 2, 5 or 10 g daily.

Possible routes of administration of the pharmaceutical compositions of the present invention include, for example, enteral (e.g., oral and rectal) and parenteral. For example, a liquid preparation may be administered, for example, orally or rectally. Additionally, a homogenous mixture can be completely dispersed in water, admixed under sterile conditions with physiologically acceptable diluents, preservatives, buffers or propellants in order to form a spray or inhalant. The route of administration will, of course, depend upon the desired effect. For example, if the composition is being utilized to treat rough, dry, or aging skin, to treat injured or burned skin, or to treat skin or hair affected by a disease or condition, it may perhaps be applied topically.

The dosage of the composition to be administered to the patient may be determined by one of ordinary skill in the art and depends upon various factors such as weight of the patient, age of the patient, immune status of the patient, etc.

With respect to form, the composition may be, for example, a solution, a dispersion, a suspension, an emulsion or a sterile powder which is then reconstituted.

The present invention also includes the treatment of various disorders by use of the pharmaceutical and/or nutritional compositions described herein. In particular, the compositions of the present invention may be used to treat restenosis after angioplasty. Furthermore, symptoms of inflammation, rheumatoid arthritis, asthma and psoriasis may also be treated with the compositions of the invention. Evidence also indicates that PUFAs may be involved in calcium metabolism; thus, the compositions of the present invention may, perhaps, be utilized in the treatment or prevention of osteoporosis and of kidney or urinary tract stones.

Additionally, the compositions of the present invention may also be used in the treatment of cancer. Malignant cells have been shown to have altered fatty acid compositions. Addition of fatty acids has been shown to slow their growth, cause cell death and increase their susceptibility to chemotherapeutic agents. Moreover, the compositions of the present invention may also be useful for treating cachexia associated with cancer.

The compositions of the present invention may also be used to treat diabetes (see U.S. Pat. No. 4,826,877 and Horrobin et al., Am. J. Clin. Nutr. Vol. 57 (Suppl.) 732S-737S). Altered fatty acid metabolism and composition have been demonstrated in diabetic animals.

Furthermore, the compositions of the present invention, comprising PUFAs produced either directly or indirectly through the use of the desaturase enzymes, may also be used in the treatment of eczema, in the reduction of blood pressure, and in the improvement of mathematics examination scores. Additionally, the compositions of the present invention may be used in inhibition of platelet aggregation, induction of vasodilation, reduction in cholesterol levels, inhibition of proliferation of vessel wall smooth muscle and fibrous tissue (Brenner et al., Adv. Exp. Med. Biol. Vol. 83, p.85-101, 1976), reduction or prevention of gastrointestinal bleeding and other side effects of non-steroidal anti-inflammatory drugs (see U.S. Pat. No. 4,666,701), prevention or treatment of endometriosis and premenstrual syndrome (see U.S. Pat. No. 4,758,592), and treatment of myalgic encephalomyelitis and chronic fatigue after viral infections (see U.S. Pat. No. 5,116,871).

Further uses of the compositions of the present invention include use in the treatment of AIDS, multiple sclerosis, and inflammatory skin disorders, as well as for maintenance of general health.

Additionally, the composition of the present invention may be utilized for cosmetic purposes. It may be added to pre-existing cosmetic compositions such that a mixture is formed or may be used as a sole composition.

Veterinary Applications

It should be noted that the above-described pharmaceutical and nutritional compositions may be utilized in connection with animals (i.e., domestic or non-domestic), as well as humans, as animals experience many of the same needs and conditions as humans. For example, the oil or acids of the present invention may be utilized in animal feed supplements, animal feed substitutes, animal vitamins or in animal topical ointments.

The present invention may be illustrated by the use of the following non-limiting examples:

### EXAMPLE I

### Design of Degenerate Oligonucleotides for the Isolation of Desaturases from Thraustochytrium aureum and cDNA Library Construction

The fatty acid composition analysis of the marine fungus Thraustochytrium aureum (T. aureum) (ATCC 34304) was investigated to determine the types and amounts of polyunsaturated fatty acids (PUFAs). This fungus had substantial amounts of longer chain PUFAs such as arachidonic acid (ARA, 20:4n-6) and eicosapentaenoic acid (EPA, 20:5 n-3). However, T. aureum also produced PUFAs such as adrenic acid (ADA, 22:4n-6), ω6-docosapentaenoic acid (ω6-DPA, 22:5n-6), ω03-docosapentaenoic acid (ω3-DPA, 22:5n-3), with the highest amount of fatty acid being docosahexaenoic acid (DHA, 22:6n-3) (see FIG. 1). Thus in addition to Δ6-, Δ5- and Δ17-desaturases, T. aureum probably contains a Δ19-desaturase which converts ADA to ω3-DPA or ω6-DPA to DHA and/or a Δ4-desaturase which desaturates ADA to ω6-DPA or ω3-DPA to DHA. The goal was therefore to attempt to isolate the predicted desaturase genes from T. aureum, and to verify the functionality of the enzymes by expression in an alternate host.

To isolate genes encoding for functional desaturase enzymes, a cDNA library was constructed. T. aureum (ATCC 34304) cells were grown in BY+ Media (#790, Difco, Detroit, Mich.) at room temperature for 4 days, in the presence of light, and with constant agitation (250 rpm) to obtain the maximum biomass. These cells were harvested by centrifugation at 5000 rpm for 10 minutes and rinsed in ice-cold RNase-free water. These cells were then lysed in a French press at 10,000 psi, and the lysed cells were directly collected into TE buffered phenol. Proteins from the cell lysate were removed by repeated phenol: chloroform (1:1 v/v) extraction, followed by a chloroform extraction. The nucleic acids from the aqueous phase were precipitated at −70° C. for 30 minutes using 0.3M (final concentration) sodium acetate (pH 5.6) and one volume of isopropanol. The precipitated nucleic acids were collected by centrifugation at 15,000 rpm for 30 minutes at 4° C., vacuum-dried for 5 minutes and then treated with DNaseI (RNase-free) in 1×DNase buffer (20 mM Tris-Cl, pH 8.0; 5 mM MgCl₂) for 15 minutes at room temperature. The reaction was quenched with 5 mM EDTA (pH 8.0) and the RNA further purified using the Qiagen RNeasy Maxi kit (Qiagen, Valencia, Calif.) as per the manufacturer's protocol.

Messenger RNA was isolated from total RNA using oligo dT cellulose resin, and the pBluescript II XR library construction kit (Stragene, La Jolla, Calif.) was used to synthesize double stranded cDNA which was then directionally cloned (5′ EcoRI/3′ XhoI) into pBluescript II SK(+) vector (Stragene, La Jolla, Calif.). The T. aureum library contained approximately 2.5×10⁶ clones each with an average insert size of approximately 700 bp. Genomic DNA from PUFA-producing T. aureum cultures was isolated by crushing the culture in liquid nitrogen and was purified using Qiagen Genomic DNA Extraction Kit (Qiagen, Valencia, Calif.).

The approach taken was to design degenerate oligonucleotides (primers) that represent amino acid motifs that are conserved in known desaturases. These primers could be then used in a PCR reaction to identify a fragment containing the conserved regions in the predicted desaturase genes from fungi. Since the only fungal desaturases which have been identified are Δ5- and Δ6-desaturase genes from Mortierella alpina (Genbank accession numbers AF067650, AB020032, respectively), desaturase sequences from plants as well as animals were taken into consideration during the design of these degenerate primers. In particular, known Δ5- and Δ6-desaturase sequences from the following organisms were used for the design of these degenerate primers: Mortierella alpina, Borago officinalis, Helianthus annuus, Brassica napus, Dictyostelium discoideum, Rattus norvegicus, Mus musculus, Homo sapiens, Caenorhabditis elegans, Arabidopsis thaliana, and Ricinus communis. The degenerate primers used were as follows using the CODEHOP Blockmaker program (http://blocks.fhcrc.org/codehop.html):

a. Protein motif 1: NH₃— VYDVTEWVKRHPGG —COOH

Primer RO 834 (SEQ ID NO: 1):

5′-GTBTAYGAYGTBACCGARTGGGTBAAGCGYCAYCCBGGHGGH-3′

B. Protein Motif 2: NH₃— GASANWWKHQHNVHH —COOH

Primer RO835 (Forward)(SEQ ID NO: 2):

5-′GGHGCYTCCGCYAACTGGTGGAAGCAYCAGCAYAACGTBCAYCAY-3′

Primer RO836 (Reverse) (SEQ ID NO: 3)

5-′RTGRTGVACGTTRTGCTGRTGCTTCCACCAGTTRGCGGARGCDCC-3′

C. Protein Motif 3: NH₃— NYQIEHHLFPTM —COOH

Primer RO838 (Reverse) (SEQ ID NO: 4)

5′-TTGATRGTCTARCTYGTRGTRGASAARGGVTGGTAC-3′

In addition, two more primers were designed based on the 2nd and 3rd conserved ‘Histidine-box’ found in known Δ6-desaturases. These were:

Primer RO753 (SEQ ID NO: 5) 5′-CATCATCATXGGRAAXARRTGRTG-3′

Primer RO754 (SEQ ID NO: 6) 5′-CTACTACTACTACAYCAYACXTAY ACXAAY-3′

The degeneracy code for the oligonucleotide sequences was:

B=C,G,T; H=A,C,T; S=C,G; R=A,G; V=A,C,G; Y=C,T; D=A,T,C; X=A,C,G,T

### EXAMPLE II

### Use of Degenerate oligonucleotides for the Isolation of a Desaturase from a Fungus

To isolate putative desaturase genes, total RNA was isolated using the lithium chloride method (Hoge, et al. (1982) Exp. Mycol. 6:225-232). Approximately 5 μg was reverse transcribed using the SuperScript Preamplification system (LifeTechnologies, Rockville, Md.) to produce first strand cDNA. The following primer combinations were used: RO834/836, RO834/838, RO835/836, RO835/838 and RO753/754 were used in several PCR reactions with different thermocycling parameters and Taq polymerase at annealing temperatures below 60° C., but no bands were produced.

In additional attempts to isolate fragments of desaturases, the degenerate primers RO834/838 (designed with the block maker program) and RO753/754 were used in a 50 μl reaction. The following components were combined: 2 μl of the first strand cDNA template, 20 mM Tris-HCl, pH 8.4, 50 mM KCl, 1.5 mM MgCl₂, 200 μM each deoxyribonucleotide triphosphate, 0.2 pmole final concentration of each primer and cDNA polymerase (Clonetech, Palo Alto, Calif.). Thermocycling was carried out as follows: an initial denaturation at 94° C. for 3 minutes, followed by 35 cycles denaturation at 94° C. for 30 seconds, annealing at 60° C. for 30 seconds and extension at 72° C. for 1 minute. This was followed by a final extension at 72° C. for 7 minutes. Two faint bands of approximately 1000 bp were detected for primers RO834/838, while a slightly smaller but more intense band of 800-900 bp was found with the primer pair RO753/754. The reactions were separated on a 1% agarose gel, excised, and purified with the QiaQuick Gel Extraction Kit (Qiagen, Valencia, Calif.). The staggered ends on these fragments were ‘filled-in’ using T4 DNA polymerase (LifeTechnologies, Rockville, Md.) as per manufacture's specifications, and these DNA fragments were cloned into the PCR-Blunt vector (Invitrogen, Carlsbad, Calif.). The recombinant plasmids were transformed into TOP10 supercompetent cells (Invitrogen, Carlsbad, Calif.) and clones were partially sequenced.

Subsequently, the sequences of clone 30-3 (reaction with RO834 /838) and clone 17-l(reaction with RO753/754) were found to overlap to create a 1313 bp fragment. The fragment was translated and Tfasta used to search the GenBank database. The highest match was Mortierella alpina Δ5-desaturase (Genbank accession # AF067654) (27% homology in 202 amino acids), Spirulina platensis Δ6-desaturase (Genbank accession number X87094) (30% homology in 121 amino acids), Dictyostelium discoideum Δ5-desaturase (Genbank accession number AB02931) (26% homology in 131 amino acids), and M. alpina Δ6-desaturase (accession number AF110510 (30% homology in 86 amino acids). Since there was a reasonable degree of amino acid homology to known desaturases, a full-length gene encoding a potential desaturase was sought to determine its activity when expressed in yeast.

### EXAMPLE III

### Isolation of the Full Length Gene Sequence from T. aureum (ATCC 34304)

To find the full-length gene, two separate PCR reactions were carried out in an attempt to determine the two ends of putative desaturase from the cDNA library. For the 3′ end of the gene, RO898 (SEQ ID NO: 7) (5′-CCCAGTCACGACGTTGTAAAACGACGGCCAG-3′) (designed based on the sequence of the pBluescript SK(+) vector (Stragene, La Jolla, Calif.) was used in a PCR amplification reaction along with a gene specific primer RO930 (SEQ ID NO: 8) (5′-GACGATTAACAAGGTGATTTCCCAGGATGTC). In this case, the Advantage -GC cDNA PCR kit (Clonetech, Palo Alto, Calif.) was used to overcome PCR amplification problems that occur with GC rich sequences (61% for 1313 bp fragment). PCR thermocycling conditions were as follows: the template was initially denatured at 94° C. for 3 minutes, followed by 30 cycles of [94° C. for 30 seconds, 52° C. for 30 seconds, and 72° C. for 1 minute], and finally an extension cycle at 72° C. for 7 minutes with 20 pmoles of each primer. The PCR products thus obtained was resolved on a 1% agarose gel, excised, and gel purified using the Qiagen Gel Extraction Kit (Qiagen, Valencia, Calif.). The staggered ends on the fragment was ‘filled-in’ using T4 DNA polymerase (LifeTechnologies, Rockville, Md.) as per manufactures specifications and cloned into the PCR-Blunt vector (Invitrogen, Carlsbad, Calif.) as described in Example II. Clone 93-3 sequence overlapped the original 1313 bp fragment and was found to contain an open reading frame, a stop codon, and a poly A tail indicating that this was the 3′ end of the gene. Two primers were designed based on clone 93-3 sequence near the stop codon with an XhoI created site (underlined) as follows: RO973 (SEQ ID NO: 9) (5′-GACTAACTCGAGTCACGTGGACCAGGCCGTGAGGTCCT-3′) and RO974 (SEQ ID NO: 10) (5′-GACTAACTCGAGTTGACGAGGTTTGTAT GTTCGGCGGTTTGCTTG-3′). Two primers were deliberately chosen because RO973, that contained the stop codon, was high in GC (60%) and might not amplify well. On the other hand, RO974, downstream of the stop codon, was much lower in GC (48%).

Following the same protocol as described above to isolate the 5′ end of the gene, RO899 (SEQ ID NO: 11) (5′-AGCGGATAACAATTTCACACAGGAAACAGC-3′) (designed based on the sequence of the pBluescript SK(+) vector) and the gene specific oligonucleotide RO1004 (SEQ ID NO: 12) (5′-TGGCTACCGTCGTGCTGGATGCAAGTTCCG-3′) were used for amplification of the cDNA library. Amplification was carried out using 10 pmols of each primer and the cDNA polymerase kit (Clonetech, Palo Alto, Calif.). The reaction conditions included an initial denaturation at 94° C. for 1 minute, followed by 30 cycles of [94° C. for 30 seconds, 68° C. for 3 minutes], and finally an extension cycle at 68° C. for 5 minutes. The PCR products thus obtained were cloned into the PCR-Blunt vector (Invitrogen, Carlsbad, Calif.) following the same protocol as described above. The recombinant plasmids were transformed into TOP10 supercompetent cells (Invitrogen, Carlsbad, Calif.), and clones were sequenced. Clone 1004-5 contained an open reading frame, several start codons, and overlapped the original 1313 bp sequence indicating that this was the 5′ end of the gene.

To isolate the full-length gene, a primer for the 5′ end of the putative desaturase was designed with a created EcoRI (underlined) as follows: RO1046 (SEQ ID NO: 13) (5′-CGCATGGAATTCATGACGGTCGGGTTTGACGAAACGGTG-3′).

To isolate a full-length clone, both RO1046/973 and RO1046/974 were used with cDNA isolated from the library and genomic DNA as a target. Both cDNA polymerase (Clonetech, Palo Alto, Calif.) and -GC Advantage Polymerase (Clonetech, Palo Alto, Calif.) were used to amplify their respective targets with 10 pmol of primer with the following reaction conditions: an initial denaturation at 94° C. for 1 minute, followed by 30 cycles of [94° C. for 30 seconds, 68° C. for 3 minutes], and finally an extension cycle at 68° C. for 5 minutes. The reactions were gel purified, cut with EcoRI/XhoI and cloned into EcoRI/XhoI prepared yeast expression vector pYX242 (Invitrogen, Carlsbad, Calif.) that had been treated with shrimp alkaline phosphatase (Roche, Indianapolis, Ind.) to prevent recircularization. Initial analysis of the full-length sequences showed several base changes. Clones 112-3 and 112-5 (designated pRTA7 and 8, respectively) were derived from the amplification with genomic DNA and -GC Advantage polymerase using primers RO1046/974. Clone 110-3 (designated pRTA5) was derived from a reaction with RO1046/973, genomic DNA target and cDNA polymerase. Clone 111-1 (designated pRTA6) was isolated from the reaction using RO1046/974, cDNA target and -GC Advantage polymerase kit. The sequence of these four plasmids, pRTA5 (SEQ ID NO: 14), pRTA6 (SEQ ID NO: 15), pRTA7 (SEQ ID NO: 16), pRTA8 (SEQ ID NO: 17) is shown in FIGS. 3-6, respectively. (Plasmids pRTA7 and pRTA8 were deposited with the American Type Culture Collection, 10801 University Boulevard, Manassas, Va. 20110 on Apr. 19, 2001 and were accorded accession numbers PTA-3301 and PTA-3300 , respectively.) This putative desaturase of 1548 bp and 515 amino acids (see FIGS. 7-10 and SEQ ID NOS: 18, 19, 20, 21, respectively) had many of the characteristics of described desaturases. The amino acids corresponding to the 5′ end of the enzyme are homologous to cytochrome b5. There are also number of histidine boxes at the following amino acids: 178-183-(Q)HDGSH; 213-219-(Q)HVLGHH; 262-265-HPWH; 271-275-HKFQH; and 452-457 (H)QIEHH. At least either an H or a Q precedes three of these histidine boxes which is unusual. Dictyostelium discoideum (Genbank accession number AB029311) has two similar boxes [(Q)HVIGHH and (H)QVVHH], while M. alpina (Genbank accession number AF067654) has (Q)HMLGHH and Synechocystis sp. only has one (H)QVTHH. The sequences of the various putative desaturases differed from each other. Several of the base changes resulted in a change in amino acid, as shown in Table 1.

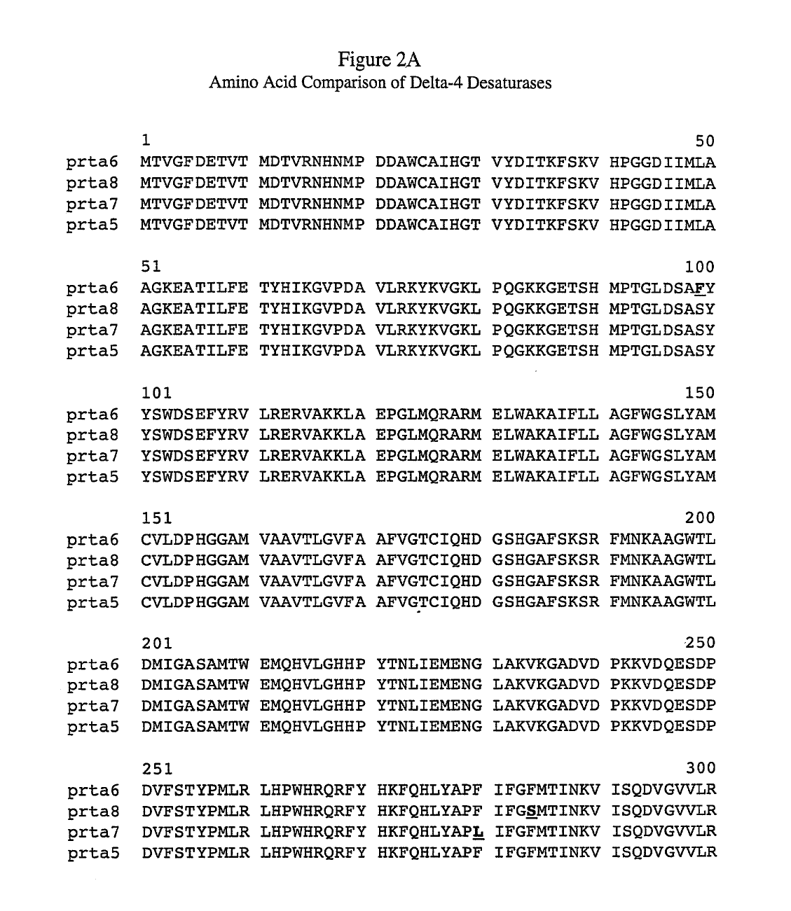

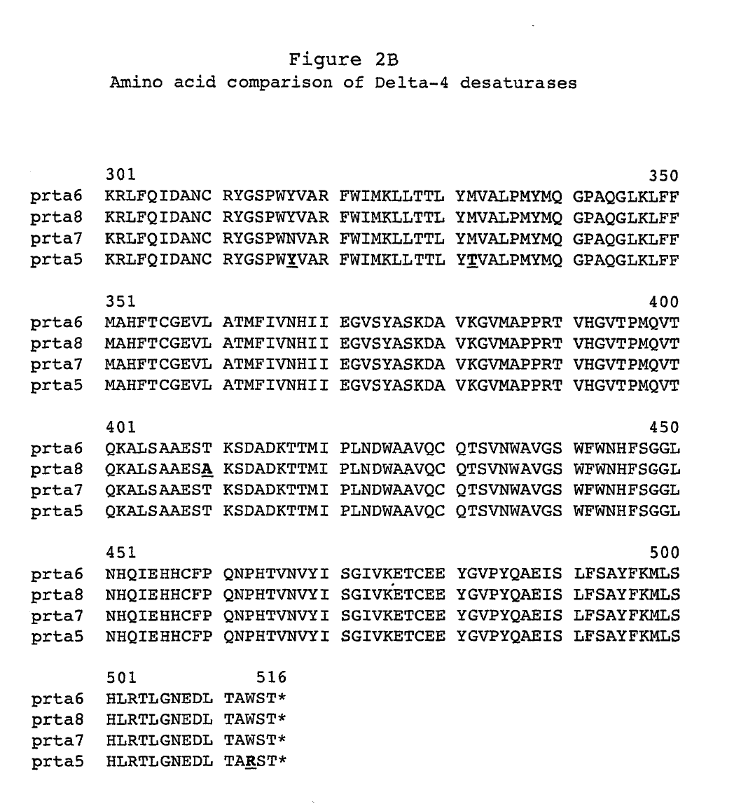

These differences could be naturally occurring variants, introduced by PCR mismatch during final amplification, or a PCR error when the initial cDNA was produced. There are 7 individual amino acid changes between the four plasmids, none of which are shared (see FIG. 2A and B, underlined and bold amino acids). These differences could alter the activity of the encoded enzyme.

TABLE 1
Amino Acid Differences in Different Clones

| Amino | | | | |
|---|---|---|---|---|
| Acid | | | | |
| Number | PRTA5 | PRTA6 | PRTA7 | PRTA8 |
| 99 | F | S | F | F |
| 280 | F | F | L | F |
| 284 | F | F | F | S |
| 317 | Y | Y | N | Y |
| 332 | T | M | M | M |
| 410 | T | T | T | A |
| 513 | R | W | W | W |

### EXAMPLE IV

### Expression of Plasmids Containing Putative Desaturases in Yeast

All four plasmids were transformed into competent Saccharomyces cerevisiae strain 334. Yeast transformation was carried out using the Alkali-Cation Yeast Transformation Kit (BIO 101, Vista, Calif.) according to conditions specified by the manufacturer. Transformants were selected for leucine auxotrophy on media lacking leucine (DOB [-Leu]). To detect the specific desaturase activity of these clones, transformants were grown in the presence of 50 μM specific fatty acid substrates as listed below:

a. Linoleic acid (18:2n-6) (conversion to alpha-linolenic acid would indicate Δ15-desaturase activity and conversion to gammα-linolenic acid would indicate Δ6-desaturase activity)

b. Alpha-linolenic acid (18:3n-3) (conversion to stearidonic acid would indicate Δ6-desaturase activity)

a. Arachidonic acid (20:4n-6) (conversion to eicosapentaenoic acid would indicate Δ17-desaturase activity)

b. Adrenic acid (22:4n-6) (conversion to ω3-docosapentaenoic acid would indicate Δ19-activity or conversion to ω6-docosapentaenoic acid would indicate Δ4-desaturase activity.

c. ω3-Docosapentaenoic acid (22:5:n-3) (conversion to docosahexaenoic would indicate Δ4-desaturase activity The negative control strain was S. cerevisiae 334 containing the unaltered pYX242 vector, and these were grown simultaneously.

The cultures were vigorously agitated (250 rpm) and grown for 48 hours a 24° C. in the presence of 50 ∥M (final concentration) of the various substrates in 50 ml of media lacking leucine after inoculation with overnight growth of single colonies in yeast peptone dextrose broth (YPD) at 30° C. The cells were pelleted, and the pellets vortexed in methanol; chloroform was added along with tritridecanoin (as an internal standard). These mixtures were incubated for at least an hour at room temperature or at 4° C. overnight. The chloroform layer was extracted and filtered through a Whatman filter with 1 gm anhydrous sodium sulfate to remove particulates and residual water. The organic solvents were evaporated at 40° C. under a stream of nitrogen. The extracted lipids were then derivitized to fatty acid methyl esters (FAME) for gas chromatography analysis (GC) by adding 2 mls of 0.5 N potassium hydroxide in methanol to a closed tube. The samples were heated to 95° C.-100° C. for 30 minutes and cooled to room temperature. Approximately 2 ml of 14% borontrifluoride in methanol was added and the heating repeated. After the extracted lipid mixture cooled, 2 ml of water and 1 ml of hexane were added to extract the fatty acid methyl esters (FAME) for analysis by GC. The percent conversion was calculated by dividing the product produced by the sum of (the product produced+the substrate added) and then multiplying by 100.

The results showed conversion of (ω-3DPA to DHA and ADA to ω6-DPA. This would indicate Δ4-desaturase activity (see Table 2).

TABLE 2
Percent Conversion of Different Substrate Concentrations to Product

| | 25 uM | 50 uM | 100 uM | 25 uM | 50 uM | 100 uM |
|---|---|---|---|---|---|---|
| Clone | 22:4n-6 | 22:4n-6 | 22:4n-6 | 22:5n-3 | 22:5n-3 | 22:5n-3 |
| PYX242 | 0 | 0 | 0 | 0 | 0 | 1.28 |
| (control) | | | | | | |
| PRTA5 | 3.91 | 0.9 | 1.24 | 10 | 6.89 | 3.1 |
| PRTA6 | 4.69 | 2.77 | 1.18 | 14.26 | 8.52 | 4.98 |
| PRTA7 | 10.97 | 6.11 | 3.14 | 36.34 | 17.52 | 9.92 |
| PRTA8 | 5.55 | 2.43 | 0.92 | 19.44 | 8.52 | 4.33 |

22:4n-6 to 22:5n-6 (Adrenic acid to ω6-Docosapentaenoic acid)
22:5n-3 to 22:6n-3 (ω3-Docosapentaenoic acid to Docosahexaenoic acid)

In particular, this is the first demonstration a Δ4-desaturase gene with in vivo expression data. The conversion for the four clones ranged from 3.91% to 10.97% for production of ω6-DPA from ADA and 10% to 36.34% for production of DHA from ω-3DPA. The enzyme appears to be much more active in the production of DHA rather than ω6-DPA, as indicated by the reduced percent conversion, 36.34% vs 10.97%, respectively, for 25 μm of substrate for clone pRTA7. At 100 μm concentration of either substrate, the percent conversion (see Table 2) as well as the amount of product produced (data not shown) decreased, indicating that there may be feedback inhibition of the desaturation step by the substrate. The amount of ω3-DPA (22:5n-3) incorporated (as a percent of the total lipid) is similar for all four plasmids (see Table 3, below). However the amount produced as a percent of the total does vary from 2.74(PRTA5) to 8.11% (PRTA7). The difference in the conversion rates and percent produced could be due to the difference in sequence, hence amino acid variation of the encoded enzyme in the four plasmids.

TABLE 3
Fatty Acid as a Percentage of Total Lipid Extracted from Yeast

| | 22:4(n-6) | 22:5(n-3) | 22:5(n-6) | 22:6(n-3) |
|---|---|---|---|---|
| Clone | Incorporated | Produced | Incorporated | Produced |
| PYX242 | 38.96 | 0 | 11.2 | 0 |
| (control) | | | | |
| PRTA5 | 14.5 | 0.59 | 19.8 | 2.74 |
| PRTA6 | 16.07 | 0.79 | 17.97 | 4.38 |
| PRTA7 | 39.88 | 4.91 | 14.21 | 8.11 |
| PRTA8 | 36.94 | 2.17 | 17.45 | 4.25 |

25 μM substrate data shown
Key:
22:4(n-6) = Adrenic acid
22:5(n-3) = ω3-Docosapentaenoic acid
22:5(n-6) = ω6-Docosapentaenoic acid
22:6(n-3) = Docosahexaenoic acid

This data shows unequivocally that these plasmids indeed encode a Δ4-desaturase, which has preferred activity on conversion of ω3-DPA to DHA activity over conversion of ADA to ω6-DPA.

### EXAMPLE V

### Expression of Δ4-desaturase with the Mouse Elongase in Yeast

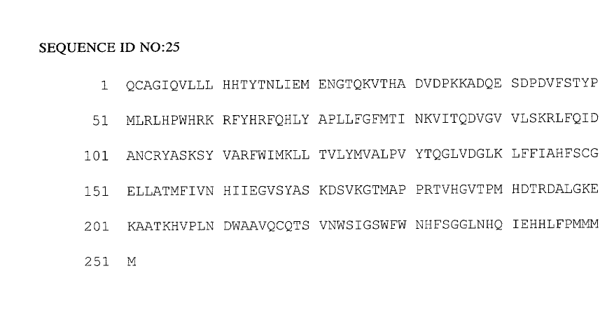

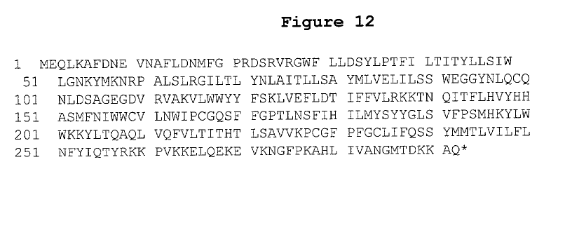

The plasmids pRTA7 and pRTA8 (which had the two highest percent conversion) may be individually co-transformed with pRMELO4, a clone that contains a mouse elongase gene from pRAE-84 (see U.S. patent application Ser. No. 09/624,670 incorporated herein in its entirety). The mouse elongase of 879 base pairs (see FIG. 12 and SEQ ID NO: 22) may be cloned as an EcoRI/SalI fragment in the yeast expression vector pYES2 (Invitrogen, Carlsbad, Calif.) at the EcoRI/XhoI sites. This elongase of 292 amino acids catalyzes several of the elongation steps in the PUFA pathway, specifically AA to ADA and EPA to ω3-DPA. ADA and ω3-DPA are substrates for the Δ4-desaturase. Yeast transformants may be selected on minimal media lacking leucine and uracil (DOB[-Leu-Ura]) for selection of Δ4-desaturase (pRTA7 or pRTA8) and pRMELO4 (mouse elongase). Growth and expression of the yeast culture containing pRMELO4 and pRTA7 or pRTA8 in minimal media lacking uracil and leucine and 2% galactose may result in elongation of exogenously added AA to ADA and Δ4 desaturation to ω6-DPA. Additionally, supplementation of EPA to the yeast minimal media may result in elongation to ω3-DPA by the elongase which may then be desaturated by the Δ4-desaturase to produce DHA as shown in FIG. 1. This has been previously demonstrated with elongases and other desaturases to produce AA and EPA (see PCT application WO 00/12720) and provides parallel experimental data to show that elongation of a substrate and subsequent desaturation can take place in vivo in an organism such as yeast and potentially other organisms. Further, the present data demonstrates the ability of the Δ4-desaturase to work with another enzyme in the PUFA biosynthetic pathway to produce either ω6-DPA or DHA from the precursors AA and EPA, respectively.

### EXAMPLE VI

### Homologue of Δ4-desaturase from Schizochytrium aggregatum ATCC 28209

In parallel to Example II, RNA was isolated by the acid phenol method from Schizochytrium aggregatum (S. aggregatum) ATCC 28209. Briefly, pellets of S. aggregatum were washed with cold deionized water and repelleted for 5 minutes at 3000 rpm. Approximatley 10 ml of TES solution (10 mM Tris-CL pH 7.5, 10 mM EDTA, and 0.5% SDS) was used to resuspend the pellet. Then 10 ml of acid phenol was added and incubation followed for one hour at 65° C. The pellet was placed on ice for 5 minutes, centrifuged for 5 minutes at 1000×g at 4° C., and the aqueous phase transferred to a new tube. An additional 10 ml of acid phenol was added to the aqueous phase, the mixture vortexed and separated as before. The aqueous phase containing the nucleic acids was transferred to a new tube. Approximately 1 ml of sodium acetate pH 5.3 and 25 ml of ice-cold ethanol were added for overnight precipitation at −70° C. The next day, the tubes were centrifuged for 15 minutes at 14,000 rpm at 4° C. and the supernatant decanted. The pellet was washed with 10 ml of 70% ethanol and centrifuged as in the previous step. The pellet was dried and resuspended in 500 ul of RNAse free deionized water. The RNA was further purified using the Qiagen RNeasy Maxikit (Qiagen, Valencia, Calif.) as per the manufacturer's protocol.

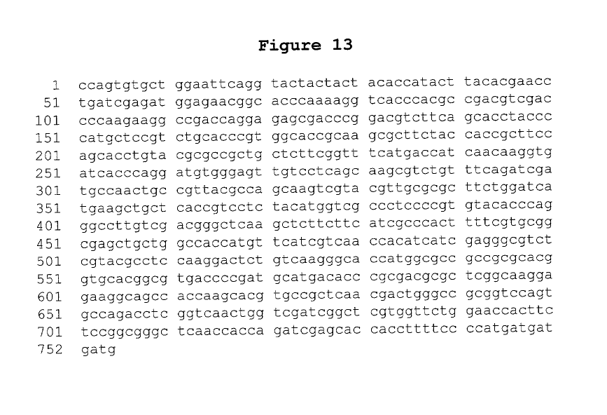

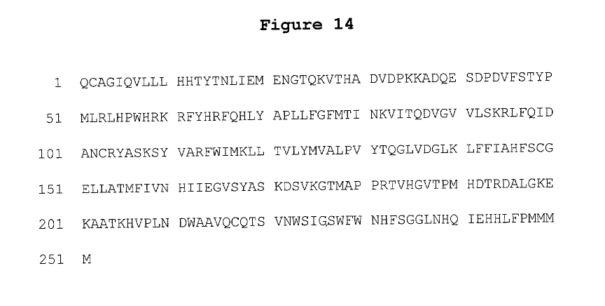

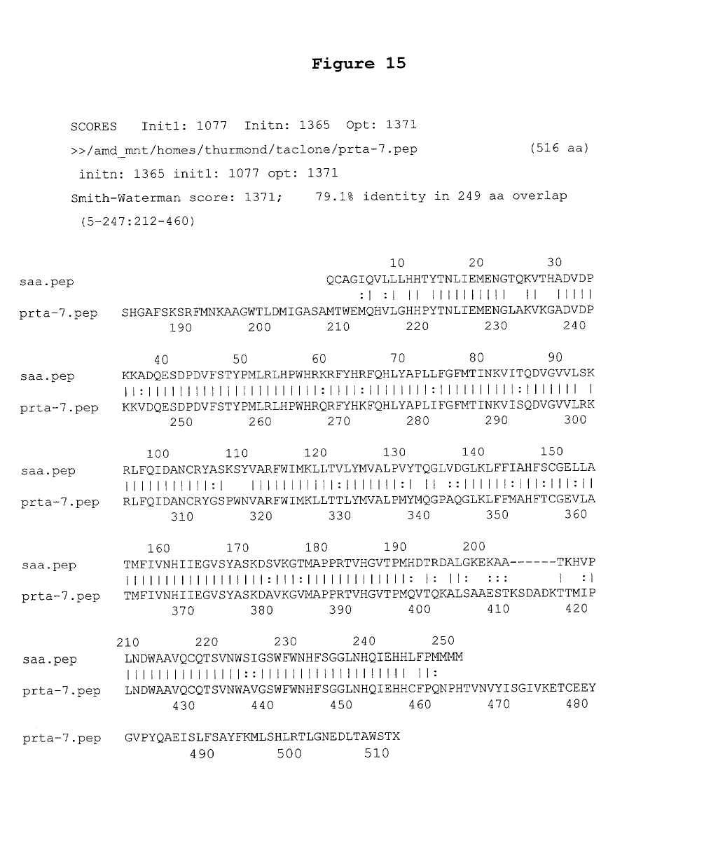

cDNA was generated using oligo dT with the SuperScript Preamplification system (Life Technologies, Rockville, Md.) with 5 ug of RNA from S. aggregatum. Since S. aggregatum produces large quantities of DHA, a Δ4-desaturase would be required for DHA production. In an identical experiment, primers RO753 (SEQ ID NO: 5) and RO754 (SEQ ID NO: 6) were used in the same reaction as in Example II to produce a band around 800 base pairs. As before the DNA generated from the PCR reaction was separated on a 1% gel, excised, purified, and cloned into the PCR-Blunt vector (Invitrogen, Carlsbad, Calif.). The DNA sequence generated from clones saa9 and saa5 overlapped to create the sequence saa.con (SEQ ID NO: 24 and FIG. 13). The translation of the open reading frame of saa.con DNA sequence to an amino acid sequence (SEQ ID NO: 25 and FIG. 14) aligned with pRTA7 is shown in FIG. 15. The amino acid sequence of the Δ4-desaturase from clone pRTA7 has 79.1% identity with the translated saa.con sequence over 249 amino acids. This sequence, due to its high identity with a known Δ4-desaturase, is most likely a fragment of a Δ4-desaturase from S. aggregatum. This example provides evidence that this procedure can be used to isolate Δ4-desaturases from other organisms.

Nutritional Compositions

The PUFAs described in the Detailed Description may be utilized in various nutritional supplements, infant formulations, nutritional substitutes and other nutritional solutions.

I. Infant Formulations

A. Isomil® Soy Formula with Iron:

Usage: As a beverage for infants, children and adults with an allergy or sensitivity to cows milk. A feeding for patients with disorders for which lactose should be avoided: lactase deficiency, lactose intolerance and galactosemia.

Features:

Soy protein isolate to avoid symptoms of cow's-milk-protein allergy or sensitivity.

Lactose-free formulation to avoid lactose-associated diarrhea.

Low osmolality (240 mOs/kg water) to reduce risk of osmotic diarrhea.

Dual carbohydrates (corn syrup and sucrose) designed to enhance carbohydrate absorption and reduce the risk of exceeding the absorptive capacity of the damaged gut.

1.8 mg of Iron (as ferrous sulfate) per 100 Calories to help prevent iron deficiency.

Recommended levels of vitamins and minerals.

Vegetable oils to provide recommended levels of essential fatty acids.

Milk-white color, milk-like consistency and pleasant aroma.

Ingredients: (Pareve) 85% water, 4.9% corn syrup, 2.6% sugar (sucrose), 2.1% soy oil, 1.9% soy protein isolate, 1.4% coconut oil, 0.15% calcium citrate, 0.11% calcium phosphate tribasic, potassium citrate, potassium phosphate monobasic, potassium chloride, mono- and disglycerides, soy lecithin, carrageenan, ascorbic acid, L-methionine, magnesium chloride, potassium phosphate dibasic, sodium chloride, choline chloride, taurine, ferrous sulfate, m-inositol, alpha-tocopheryl acetate, zinc sulfate, L-carnitine, niacinamide, calcium pantothenate, cupric sulfate, vitamin A palmitate, thiamine chloride hydrochloride, riboflavin, pyridoxine hydrochloride, folic acid, manganese sulfate, potassium iodide, phylloquinone, biotin, sodium selenite, vitamin D3 and cyanocobalamin.

B. Isomil® DF Soy Formula For Diarrhea:

Usage: As a short-term feeding for the dietary management of diarrhea in infants and toddlers.

Features:

First infant formula to contain added dietary fiber from soy fiber specifically for diarrhea management.

Clinically shown to reduce the duration of loose, watery stools during mild to severe diarrhea in infants.

Nutritionally complete to meet the nutritional needs of the infant.

Soy protein isolate with added L-methionine meets or exceeds an infant's requirement for all essential amino acids.

Lactose-free formulation to avoid lactose-associated diarrhea.

Low osmolality (240 mOsm/kg water) to reduce the risk of osmotic diarrhea.

Dual carbohydrates (corn syrup and sucrose) designed to enhance carbohydrate absorption and reduce the risk of exceeding the absorptive capacity of the damaged gut.

Meets or exceeds the vitamin and mineral levels recommended by the Committee on Nutrition of the American Academy of Pediatrics and required by the Infant Formula Act.

1.8 mg of iron (as ferrous sulfate) per 100 Calories to help prevent iron deficiency.

Vegetable oils to provide recommended levels of essential fatty acids.

Ingredients: (Pareve) 86% water, 4.8% corn syrup, 2.5% sugar (sucrose), 2.1% soy oil, 2.0% soy protein isolate, 1.4% coconut oil, 0.77% soy fiber, 0.12% calcium citrate, 0.11% calcium phosphate tribasic, 0.10% potassium citrate, potassium chloride, potassium phosphate monobasic, mono and diglycerides, soy lecithin, carrageenan, magnesium chloride, ascorbic acid, L-methionine, potassium phosphate dibasic, sodium chloride, choline chloride, taurine, ferrous sulfate, m-inositol, alpha-tocopheryl acetate, zinc sulfate, L-carnitine, niacinamide, calcium pantothenate, cupric sulfate, vitamin A palmitate, thiamine chloride hydrochloride, riboflavin, pyridoxine hydrochloride, folic acid, manganese sulfate, potassium iodide, phylloquinone, biotin, sodium selenite, vitamin D3 and cyanocobalamin.

C. Isomil® SF Sucrose-free Soy Formula with Iron:

Usage: As a beverage for infants, children and adults with an allergy or sensitivity to cow's-milk protein or an intolerance to sucrose. A feeding for patients with disorders for which lactose and sucrose should be avoided.

Features:

Soy protein isolate to avoid symptoms of cow's-milk-protein allergy or sensitivity.

Lactose-free formulation to avoid lactose-associated diarrhea (carbohydrate source is Polycose® Glucose Polymers).

Sucrose free for the patient who cannot tolerate sucrose.

Low osmolality (180 mOsm/kg water) to reduce risk of osmotic diarrhea.

1.8 mg of iron (as ferrous sulfate) per 100 Calories to help prevent iron deficiency.

Recommended levels of vitamins and minerals.

Vegetable oils to provide recommended levels of essential fatty acids.

Milk-white color, milk-like consistency and pleasant aroma.

Ingredients: (Pareve) 75% water, 11.8% hydrolized cornstarch, 4.1% soy oil, 4.1% soy protein isolate, 2.8% coconut oil, 1.0% modified cornstarch, 0.38% calcium phosphate tribasic, 0.17% potassium citrate, 0.13% potassium chloride, mono- and diglycerides, soy lecithin, magnesium chloride, abscorbic acid, L-methionine, calcium carbonate, sodium chloride, choline chloride, carrageenan, taurine, ferrous sulfate, m-inositol, alpha-tocopheryl acetate, zinc sulfate,L-carnitine, niacinamide, calcium pantothenate, cupric sulfate, vitamin A palmitate, thiamine chloride hydrochloride, riboflavin, pyridoxine hydrochloride, folic acid, manganese sulfate, potassium iodide, phylloquinone, biotin, sodium selenite, vitamin D3 and cyanocobalamin.

D. Isomil® 20 Soy Formula with Iron Ready to Feed, 20 Cal/fl oz.:

Usage: When a soy feeding is desired.

Ingredients: (Pareve) 85% water, 4.9% corn syrup, 2.6% sugar(sucrose), 2.1% soy oil, 1.9% soy protein isolate, 1.4% coconut oil, 0.15% calcium citrate, 0.11% calcium phosphate tribasic, potassium citrate, potassium phosphate monobasic, potassium chloride, mono- and diglycerides, soy lecithin, carrageenan, abscorbic acid, L-methionine, magnesium chloride, potassium phosphate dibasic, sodium chloride, choline chloride, taurine, ferrous sulfate, m-inositol, alpha-tocopheryl acetate, zinc sulfate, L-carnitine, niacinamide, calcium pantothenate, cupric sulfate, vitamin A palmitate, thiamine chloride hydrochloride, riboflavin, pyridoxine hydrochloride, folic acid, manganese sulfate, potassium iodide, phylloquinone, biotin, sodium selenite, vitamin D3 and cyanocobalamin.

E. Similac® Infant Formula:

Usage: When an infant formula is needed: if the decision is made to discontinue breastfeeding before age 1 year, if a supplement to breastfeeding is needed or as a routine feeding if breastfeeding is not adopted.

Features:

Protein of appropriate quality and quantity for good growth; heat-denatured, which reduces the risk of milk-associated enteric blood loss.

Fat from a blend of vegetable oils (doubly homogenized), providing essential linoleic acid that is easily absorbed.

Carbohydrate as lactose in proportion similar to that of human milk.

Low renal solute load to minimize stress on developing organs.

Powder, Concentrated Liquid and Ready To Feed forms.

Ingredients: (-D) Water, nonfat milk, lactose, soy oil, coconut oil, mono- and diglycerides, soy lecithin, abscorbic acid, carrageenan, choline chloride, taurine, m-inositol, alpha-tocopheryl acetate, zinc sulfate, niacinamide, ferrous sulfate, calcium pantothenate, cupric sulfate, vitamin A palmitate, thiamine chloride hydrochloride, riboflavin, pyridoxine hydrochloride, folic acid, manganese sulfate, phylloquinone, biotin, sodium selenite, vitamin D3 and cyanocobalamin.

F. Similac® NeoCare Premature Infant Formula with Iron:

Usage: For premature infants' special nutritional needs after hospital discharge. Similac NeoCare is a nutritionally complete formula developed to provide premature infants with extra calories, protein, vitamins and minerals needed to promote catch-up growth and support development.

Features:

Reduces the need for caloric and vitamin supplementation. More calories (22 Cal/fl oz) than standard term formulas (20 Cal/fl oz).

Highly absorbed fat blend, with medium-chain triglycerides (MCToil) to help meet the special digestive needs of premature infants.

Higher levels of protein, vitamins and minerals per 100 calories to extend the nutritional support initiated in-hospital.

More calcium and phosphorus for improved bone mineralization.

Ingredients: -D Corn syrup solids, nonfat milk, lactose, whey protein concentrate, soy oil, high-oleic safflower oil, fractionated coconut oil (medium chain triglycerides), coconut oil, potassium citrate, calcium phosphate tribasic, calcium carbonate, ascorbic acid, magnesium chloride, potassium chloride, sodium chloride, taurine, ferrous sulfate, m-inositol, choline chloride, ascorbyl palmitate, L-carnitine, alpha-tocopheryl acetate, zinc sulfate, niacinamide, mixed tocopherols, sodium citrate, calcium pantothenate, cupric sulfate, thiamine chloride hydrochloride, vitamin A palmitate, beta carotene, riboflavin, pyridoxine hydrochloride, folic acid, manganese sulfate, phylloquinone, biotin, sodium selenite, vitamin D3 and cyanocobalamin.

G. Similac Natural Care Low-iron Human Milk Fortifier Ready to Use, 24 Cal/fl oz.:

Usage: Designed to be mixed with human milk or to be fed alternatively with human milk to low-birth-weight infants.

Ingredients: -D Water, nonfat milk, hydrolyzed cornstarch, lactose, fractionated coconut oil (medium-chain triglycerides), whey protein concentrate, soy oil, coconut oil, calcium phosphate tribasic, potassium citrate, magnesium chloride, sodium citrate, ascorbic acid, calcium carbonate, mono and diglycerides, soy lecithin, carrageenan, choline chloride, m-inositol, taurine, niacinamide, L-carnitine, alpha tocopheryl acetate, zinc sulfate, potassium chloride, calcium pantothenate, ferrous sulfate, cupric sulfate, riboflavin, vitamin A palmitate, thiamine chloride hydrochloride, pyridoxine hydrochloride, biotin, folic acid, manganese sulfate, phylloquinone, vitamin D3, sodium selenite and cyanocobalamin.

Various PUFAs of this invention can be substituted and/or added to the infant formulae described above and to other infant formulae known to those in the art.

II. Nutritional Formulations

A. Ensure®

Usage: ENSURE is a low-residue liquid food designed primarily as an oral nutritional supplement to be used with or between meals or, in appropriate amounts, as a meal replacement. ENSURE is lactose- and gluten-free, and is suitable for use in modified diets, including low-cholesterol diets. Although it is primarily an oral supplement, it can be fed by tube.

Patient Conditions:

For patients on modified diets

For elderly patients at nutrition risk

For patients with involuntary weight loss

For patients recovering from illness or surgery

For patients who need a low-residue diet

Ingredients:—D Water, Sugar (Sucrose), Maltodextrin (Corn), Calcium and Sodium Caseinates, High-Oleic Safflower Oil, Soy Protein Isolate, Soy Oil, Canola Oil, Potassium Citrate, Calcium Phosphate Tribasic, Sodium Citrate, Magnesium Chloride, Magnesium Phosphate Dibasic, Artificial Flavor, Sodium Chloride, Soy Lecithin, Choline Chloride, Ascorbic Acid, Carrageenan, Zinc Sulfate, Ferrous Sulfate, Alpha-Tocopheryl Acetate, Gellan Gum, Niacinamide, Calcium Pantothenate, Manganese Sulfate, Cupric Sulfate, Vitamin A Palmitate, Thiamine Chloride Hydrochloride, Pyridoxine Hydrochloride, Riboflavin, Folic Acid, Sodium Molybdate, Chromium Chloride, Biotin, Potassium Iodide, Sodium Selenate.

B. Ensure® Bars:

Usage: ENSURE BARS are complete, balanced nutrition for supplemental use between or with meals. They provide a delicious, nutrient-rich alternative to other snacks. ENSURE BARS contain <1 g lactose/bar, and Chocolate Fudge Brownie flavor is gluten-free. (Honey Graham Crunch flavor contains gluten.)

Patient Conditions:

For patients who need extra calories, protein, vitamins and minerals.

Especially useful for people who do not take in enough calories and nutrients.

For people who have the ability to chew and swallow

Not to be used by anyone with a peanut allergy or any type of allergy to nuts.

Ingredients: Honey Graham Crunch—High-Fructose Corn Syrup, Soy Protein Isolate, Brown Sugar, Honey, Maltodextrin (Corn), Crisp Rice (Milled Rice, Sugar [Sucrose], Salt [Sodium Chloride] and Malt), Oat Bran, Partially Hydrogenated Cottonseed and Soy Oils, Soy Polysaccharide, Glycerine, Whey Protein Concentrate, Polydextrose, Fructose, Calcium Caseinate, Cocoa Powder, Artificial Flavors, Canola Oil, High-Oleic Safflower Oil, Nonfat Dry Milk, Whey Powder, Soy Lecithin and Corn Oil. Manufactured in a facility that processes nuts.

Vitamins and Minerals: Calcium Phosphate Tribasic, Potassium Phosphate Dibasic, Magnesium Oxide, Salt (Sodium Chloride), Potassium Chloride, Ascorbic Acid, Ferric Orthophosphate, Alpha-Tocopheryl Acetate, Niacinamide, Zinc Oxide, Calcium Pantothenate, Copper Gluconate, Manganese Sulfate, Riboflavin, Beta Carotene, Pyridoxine Hydrochloride, Thiamine Mononitrate, Folic Acid, Biotin, Chromium Chloride, Potassium Iodide, Sodium Selenate, Sodium Molybdate, Phylloquinone, Vitamin D3 and Cyanocobalamin.

Protein: Honey Graham Crunch—The protein source is a blend of soy protein isolate and milk proteins.

| | |
|---|---|
| Soy protein isolate | 74% |
| Milk proteins | 26% |

Fat: Honey Graham Crunch—The fat source is a blend of partially hydrogenated cottonseed and soybean, canola, high oleic safflower, oils, and soy lecithin.

| | |
|---|---|
| Partially hydrogenated cottonseed and soybean oil | 76% |
| Canola oil | 8% |
| High-oleic safflower oil | 8% |
| Corn oil | 4% |
| Soy lecithin | 4% |

Carbohydrate: Honey Graham Crunch—The carbohydrate source is a combination of high-fructose corn syrup, brown sugar, maltodextrin, honey, crisp rice, glycerine, soy polysaccharide, and oat bran.

| | |
|---|---|
| High-fructose corn syrup | 24% |
| Brown sugar | 21% |
| Maltodextrin | 12% |
| Honey | 11% |
| Crisp rice | 9% |
| Glycerine | 9% |
| Soy Polysaccharide | 7% |
| Oat bran | 7% |

C. Ensure® High Protein:

Usage: ENSURE HIGH PROTEIN is a concentrated, high-protein liquid food designed for people who require additional calories, protein, vitamins, and minerals in their diets. It can be used as an oral nutritional supplement with or between meals or, in appropriate amounts, as a meal replacement. ENSURE HIGH PROTEIN is lactose- and gluten-free, and is suitable for use by people recovering from general surgery or hip fractures and by patients at risk for pressure ulcers.

Patient Conditions:

For patients who require additional calories, protein, vitamins, and minerals, such as patients recovering from general surgery or hip fractures, patients at risk for pressure ulcers, and patients on low-cholesterol diets.

Features:

Low in saturated fat

Contains 6 g of total fat and <5 mg of cholesterol per serving

Rich, creamy taste

Excellent source of protein, calcium, and other essential vitamins and minerals

For low-cholesterol diets

Lactose-free, easily digested

Ingredients:

Vanilla Supreme: -D Water, Sugar (Sucrose), Maltodextrin (Corn), Calcium and Sodium Caseinates, High-OIeic Safflower Oil, Soy Protein Isolate, Soy Oil, Canola Oil, Potassium Citrate, Calcium Phosphate Tribasic, Sodium Citrate, Magnesium Chloride, Magnesium Phosphate Dibasic, Artificial Flavor, Sodium Chloride, Soy Lecithin, Choline Chloride, Ascorbic Acid, Carrageenan, Zinc Sulfate, Ferrous Suffate, Alpha-Tocopheryl Acetate, Gellan Gum, Niacinamide, Calcium Pantothenate, Manganese Sulfate, Cupric Sulfate, Vitamin A Palmitate, Thiamine Chloride Hydrochloride, Pyridoxine Hydrochloride, Riboflavin, Folic Acid, Sodium Molybdate, Chromium Chloride, Biotin, Potassium Iodide, Sodium Selenate, Phylloquinone, Vitamin D3 and Cyanocobalamin.

Protein:

The protein source is a blend of two high-biologic-value proteins: casein and soy.

| | |
|---|---|
| Sodium and calcium caseinates | 85% |
| Soy protein isolate | 15% |

Fat:

The fat source is a blend of three oils: high-oleic safflower, canola, and soy.

| | |
|---|---|
| High-oleic safflower oil | 40% |
| Canola oil | 30% |
| Soy oil | 30% |

The level of fat in ENSURE HIGH PROTEIN meets American Heart Association (AHA) guidelines. The 6 grams of fat in ENSURE HIGH PROTEIN represent 24% of the total calories, with 2.6% of the fat being from saturated fatty acids and 7.9% from polyunsaturated fatty acids. These values are within the AHA guidelines of <30% of total calories from fat, <10% of the calories from saturated fatty acids, and <10% of total calories from polyunsaturated fatty acids.

Carbohydrate:

ENSURE HIGH PROTEIN contains a combination of maltodextrin and sucrose. The mild sweetness and flavor variety (vanilla supreme, chocolate royal, wild berry, and banana), plus VARI-FLAVORS® Flavor Pacs in pecan, cherry, strawberry, lemon, and orange, help to prevent flavor fatigue and aid in patient compliance.

Vanilla and other nonchocolate flavors:

| | |
|---|---|
| Sucrose | 60% |
| Maltodextrin | 40% |
| Chocolate: | |
| Sucrose | 70% |
| Maltodextrin | 30% |

D. Ensure® Light

Usage: ENSURE LIGHT is a low-fat liquid food designed for use as an oral nutritional supplement with or between meals. ENSURE LIGHT is lactose- and gluten-free, and is suitable for use in modified diets, including low-cholesterol diets.

Patient Conditions:

For normal-weight or overweight patients who need extra nutrition in a supplement that contains 50% less fat and 20% fewer calories than ENSURE.

For healthy adults who do not eat right and need extra nutrition.

Features:

Low in fat and saturated fat

Contains 3 g of total fat per serving and <5 mg cholesterol

Rich, creamy taste

Excellent source of calcium and other essential vitamins and minerals

For low-cholesterol diets

Lactose-free, easily digested

Ingredients:

French Vanilla: -D Water, Maltodextrin (Corn), Sugar (Sucrose), Calcium Caseinate, High-Oleic Safflower Oil, Canola Oil, Magnesium Chloride, Sodium Citrate, Potassium Citrate, Potassium Phosphate Dibasic, Magnesium Phosphate Dibasic, Natural and Artificial Flavor, Calcium Phosphate Tribasic, Cellulose Gel, Choline Chloride, Soy Lecithin, Carrageenan, Salt (Sodium Chloride), Ascorbic Acid, Cellulose Gum, Ferrous Sulfate, Alpha-Tocopheryl Acetate, Zinc Sulfate, Niacinamide, Manganese Sulfate, Calcium Pantothenate, Cupric Sulfate, Thiamine Chloride Hydrochloride, Vitamin A Palmitate, Pyridoxine Hydrochloride, Riboflavin, Chromium Chloride, Folic Acid, Sodium Molybdate, Biotin, Potassium Iodide, Sodium Selenate, Phylloquinone, Vitamin D3 and Cyanocobalamin.

Protein:

The protein source is calcium caseinate.

Calcium caseinate 100%

Fat:

The fat source is a blend of two oils: high-oleic safflower and canola.

| | |
|---|---|
| High-oleic safflower oil | 70% |
| Canola oil | 30% |

The level of fat in ENSURE LIGHT meets American Heart Association (AHA) guidelines. The 3 grams of fat in ENSURE LIGHT represent 13.5% of the total calories, with 1.4% of the fat being from saturated fatty acids and 2.6% from polyunsaturated fatty acids. These values are within the AHA guidelines of <30% of total calories from fat, <10% of the, calories from saturated fatty acids, and <10% of total calories from polyunsaturated fatty acids.

Carbohydrate:

ENSURE LIGHT contains a combination of maltodextrin and sucrose. The chocolate flavor contains corn syrup as well. The mild sweetness and flavor variety (French vanilla, chocolate supreme, strawberry swirl), plus VARI-FLAVORS® Flavor Pacs in pecan, cherry, strawberry, lemon, and orange, help to prevent flavor fatigue and aid in patient compliance.

| Vanilla and other nonchocolate flavors: | |
|---|---|
| Sucrose | 51% |
| Maltodextrin | 49% |
| Chocolate: | |
| Sucrose | 47.0% |
| Corn Syrup | 26.5% |
| Maltodextrin | 26.5% |

Vitamins and Minerals:

An 8-fl-oz serving of ENSURE LIGHT provides at least 25% of the RDIs for 24 key vitamins and minerals.

Caffeine:

Chocolate flavor contains 2.1 mg caffeine/8 fl oz.

E. Ensure Plus®

Usage: ENSURE PLUS is a high-calorie, low-residue liquid food for use when extra calories and nutrients, but a normal concentration of protein, are needed. It is designed primarily as an oral nutritional supplement to be used with or between meals or, in appropriate amounts, as a meal replacement. ENSURE PLUS is lactose- and gluten-free. Although it is primarily an oral nutritional supplement, it can be fed by tube.

Patient Conditions:

For patients who require extra calories and nutrients, but a normal concentration of protein, in a limited volume.

For patients who need to gain or maintain healthy weight.

Features:

Rich, creamy taste

Good source of essential vitamins and minerals

Ingredients:

Vanilla: -D Water, Corn Syrup, Maltodextrin (Corn), Corn Oil, Sodium and Calcium Caseinates, Sugar (Sucrose), Soy Protein Isolate, Magnesium Chloride, Potassium Citrate, Calcium Phosphate Tribasic, Soy Lecithin, Natural and Artificial Flavor, Sodium Citrate, Potassium Chloride, Choline Chloride, Ascorbic Acid, Carrageenan, Zinc Sulfate, Ferrous Sulfate, Alpha-Tocopheryl Acetate, Niacinamide, Calcium Pantothenate, Manganese Sulfate, Cupric Sulfate, Thiamine Chloride Hydrochloride, Pyridoxine Hydrochloride, Riboflavin, Vitamin A Palmitate, Folic Acid, Biotin, Chromium Chloride, Sodium Molybdate, Potassium Iodide, Sodium Selenite, Phylloquinone, Cyanocobalamin and Vitamin D3.

| Protein: | |
|---|---|
| The protein source is a blend of two high- | |
| biologic-value proteins: casein and soy. | |
| Sodium and calcium caseinates | 84% |
| Soy protein isolate | 16% |
| Fat: | |
| The fat source is corn oil. | |
| Corn oil | 100% |

Carbohydrate:

ENSURE PLUS contains a combination of maltodextrin and sucrose. The mild sweetness and flavor variety (vanilla, chocolate, strawberry, coffee, buffer pecan, and eggnog), plus VARI-FLAVORS® Flavor Pacs in pecan, cherry, strawberry, lemon, and orange, help to prevent flavor fatigue and aid in patient compliance.

| Vanilla, strawberry, butter pecan, and coffee flavors: | |
|---|---|
| Corn Syrup | 39% |
| Maltodextrin | 38% |
| Sucrose | 23% |
| Chocolate and eggnog flavors: | |
| Corn Syrup | 36% |
| Maltodextrin | 34% |
| Sucrose | 30% |

Vitamins and Minerals:

An 8-fl-oz serving of ENSURE PLUS provides at least 15% of the RDIs for 25 key Vitamins and minerals.

Caffeine:

Chocolate flavor contains 3.1 mg Caffeine/8 fl oz. Coffee flavor contains a trace amount of caffeine.

F. Ensure Plus® HN

Usage: ENSURE PLUS HN is a nutritionally complete high-calorie, high-nitrogen liquid food designed for people with higher calorie and protein needs or limited volume tolerance. It may be used for oral supplementation or for total nutritional support by tube. ENSURE PLUS HN is lactose- and gluten-free.

Patient Conditions:

For patients with increased calorie and protein needs, such as following surgery or injury.

For patients with limited volume tolerance and early satiety.

Features:

For supplemental or total nutrition

For oral or tube feeding

1.5 CaVmL,

High nitrogen

Calorically dense

Ingredients:

Vanilla: -D Water, Maltodextrin (Corn), Sodium and Calcium Caseinates, Corn Oil, Sugar (Sucrose), Soy Protein Isolate, Magnesium Chloride, Potassium Citrate, Calcium Phosphate Tribasic, Soy Lecithin, Natural and Artificial Flavor, Sodium Citrate, Choline Chloride, Ascorbic Acid, Taurine, L-Carnitine, Zinc Sulfate, Ferrous Sulfate, Alpha-Tocopheryl Acetate, Niacinamide, Carrageenan, Calcium Pantothenate, Manganese Sulfate, Cupric Sulfate, Thiamine Chloride Hydrochloride, Pyridoxine Hydrochloride, Riboflavin, Vitamin A Palmitate, Folic Acid, Biotin, Chromium Chloride, Sodium Molybdate, Potassium Iodide, Sodium Selenite, Phylloquinone, Cyanocobalamin and Vitamin D3.

G. Ensure® Powder:

Usage: ENSURE POWDER (reconstituted with water) is a low-residue liquid food designed primarily as an oral nutritional supplement to be used with or between meals. ENSURE POWDER is lactose- and gluten-free, and is suitable for use in modified diets, including low-cholesterol diets.

Patient Conditions:

For patients on modified diets

For elderly patients at nutrition risk

For patients recovering from illness/surgery

For patients who need a low-residue diet

Features:

Convenient, easy to mix

Low in saturated fat

Contains 9 g of total fat and <5 mg of cholesterol per serving

High in vitamins and minerals

For low-cholesterol diets

Lactose-free, easily digested

Ingredients: -D Corn Syrup, Maltodextrin (Corn), Sugar (Sucrose), Corn Oil, Sodium and Calcium Caseinates, Soy Protein Isolate, Artificial Flavor, Potassium Citrate, Magnesium Chloride, Sodium Citrate, Calcium Phosphate Tribasic, Potassium Chloride, Soy Lecithin, Ascorbic Acid, Choline Chloride, Zinc Sulfate, Ferrous Sulfate, Alpha-Tocopheryl Acetate, Niacinamide, Calcium Pantothenate, Manganese Sulfate, Thiamine Chloride Hydrochloride, Cupric Sulfate, Pyridoxine Hydrochloride, Riboflavin, Vitamin A Palmitate, Folic Acid, Biotin, Sodium Molybdate, Chromium Chloride, Potassium Iodide, Sodium Selenate, Phylloquinone, Vitamin D3 and Cyanocobalamin.

Protein:

The protein source is a blend of two high-biologic-value proteins: casein and soy.

| Sodium and calcium caseinates | 84% |
|---|---|
| Soy protein isolate | 16% |
| Fat: | |
| The fat source is corn oil. | |
| Corn oil | 100% |

Carbohydrate:

ENSURE POWDER contains a combination of corn syrup, maltodextrin, and sucrose. The mild sweetness of ENSURE POWDER, plus VARI-FLAVORS Flavor Pacs in pecan, cherry, strawberry, lemon, and orange, helps to prevent flavor fatigue and aid in patient compliance.

| Vanilla: | |
|---|---|
| Corn Syrup | 35% |
| Maltodextrin | 35% |
| Sucrose | 30% |

H. Ensure® Pudding

Usage: ENSURE PUDDING is a nutrient-dense supplement providing balanced nutrition in a nonliquid form to be used with or between meals. It is appropriate for consistency-modified diets (e.g., soft, pureed, or full liquid) or for people with swallowing impairments. ENSURE PUDDING is gluten-free.

Patient Conditions:

For patients on consistency-modified diets (e.g., soft, pureed, or full liquid)

For patients with swallowing impairments

Features:

Rich and creamy, good taste

Good source of essential vitamins and minerals

Convenient-needs no refrigeration

Gluten-free

Nutrient Profile per 5 oz: Calories 250, Protein 10.9%, Total Fat 34.9%, Carbohydrate 54.2%

Ingredients:

Vanilla: -D Nonfat Milk, Water, Sugar (Sucrose), Partially Hydrogenated Soybean Oil, Modified Food Starch, Magnesium Sulfate, Sodium Stearoyl Lactylate, Sodium Phosphate Dibasic, Artificial Flavor, Ascorbic Acid, Zinc Sulfate, Ferrous Sulfate, Alpha-Tocopheryl Acetate, Choline Chloride, Niacinamide, Manganese Sulfate, Calcium Pantothenate, FD&C Yellow #5, Potassium Citrate, Cupric Sulfate, Vitamin A Palmitate, Thiamine Chloride Hydrochloride, Pyridoxine Hydrochloride, Riboflavin, FD&C Yellow #6, Folic Acid, Biotin, Phylloquinone, Vitamin D3 and Cyanocobalamin.

| Protein: | |
|---|---|
| The protein source is nonfat milk. | |
| Nonfat milk | 100% |
| Fat: | |
| The fat source is hydrogenated soybean oil. | |
| Hydrogenated soybean oil | 100% |

Carbohydrate:

ENSURE PUDDING contains a combination of sucrose and modified food starch. The mild sweetness and flavor variety (vanilla, chocolate, butterscotch, and tapioca) help prevent flavor fatigue. The product contains 9.2 grams of lactose per serving.

| Vanilla and other nonchocolate flavors: | |
|---|---|
| Sucrose | 56% |
| Lactose | 27% |
| Modified food starch | 17% |
| Chocolate: | |
| Sucrose | 58% |
| Lactose | 26% |
| Modified food starch | 16% |

I. Ensure® with Fiber:

Usage: ENSURE WITH FIBER is a fiber-containing, nutritionally complete liquid food designed for people who can benefit from increased dietary fiber and nutrients. ENSURE WITH FIBER is suitable for people who do not require a low-residue diet. It can be fed orally or by tube, and can be used as a nutritional supplement to a regular diet or, in appropriate amounts, as a meal replacement. ENSURE WITH FIBER is lactose- and gluten-free, and is suitable for use in modified diets, including low-cholesterol diets.

Patient Conditions:

For patients who can benefit from increased dietary fiber and nutrients

Features:

New advanced formula-low in saturated fat, higher in vitamins and minerals

Contains 6 g of total fat and <5 mg of cholesterol per serving

Rich, creamy taste

Good source of fiber

Excellent source of essential vitamins and minerals

For low-cholesterol diets

Lactose- and gluten-free

Ingredients:

Vanilla: -D Water; Maltodextrin (Corn), Sugar (Sucrose), Sodium and Calcium Caseinates, Oat Fiber, High-Oleic Safflower Oil, Canola Oil, Soy Protein Isolate, Corn Oil, Soy Fiber, Calcium Phosphate Tribasic, Magnesium Chloride, Potassium Citrate, Cellulose Gel, Soy Lecithin, Potassium Phosphate Dibasic, Sodium Citrate, Natural and Artificial Flavors, Choline Chloride, Magnesium Phosphate, Ascorbic Acid, Cellulose Gum, Potassium Chloride, Carrageenan, Ferrous Sulfate, Alpha-Tocopheryl Acetate, Zinc Sulfate, Niacinamide, Manganese Sulfate, Calcium Pantothenate, Cupric Sulfate, Vitamin A Palmitate, Thiamine Chloride Hydrochloride, Pyridoxine Hydrochloride, Riboflavin, Folic Acid, Chromium Chloride, Biotin, Sodium Molybdate, Potassium Iodide, Sodium Selenate, Phylloquinone, Vitamin D3 and Cyanocobalamin.

| Protein: | |
|---|---|
| The protein source is a blend of two high | |
| -biologic-value proteins-casein and soy. | |
| Sodium and calcium caseinates | 80% |
| Soy protein isolate | 20% |
| Fat: | |
| The fat source is a blend of three oils: high- | |
| oleic safflower, canola, and corn. | |
| High-oleic safflower oil | 40% |
| Canola oil | 40% |
| Corn oil | 20% |

The level of fat in ENSURE WITH FIBER meets American Heart Association (AHA) guidelines. The 6 grams of fat in ENSURE WITH FIBER represent 22% of the total calories, with 2.01% of the fat being from saturated fatty acids and 6.7% from polyunsaturated fatty acids. These values are within the AHA guidelines of ≦30% of total calories from fat, <10% of the calories from saturated fatty acids, and ≦10% of total calories from polyunsaturated fatty acids.

Carbohydrate:

ENSURE WITH FIBER contains a combination of maltodextrin and sucrose. The mild sweetness and flavor variety (vanilla, chocolate, and butter pecan), plus VARI-FLAVORS® Flavor Pacs in pecan, cherry, strawberry, lemon, and orange, help to prevent flavor fatigue and aid in patient compliance.

| Vanilla and other nonchocolate flavors: | |
|---|---|
| Maltodextrin | 66% |
| Sucrose | 25% |
| Oat Fiber | 7% |
| Soy Fiber | 2% |
| Chocolate: | |
| Maltodextrin | 55% |
| Sucrose | 36% |
| Oat Fiber | 7% |
| Soy Fiber | 2% |

Fiber:

The fiber blend used in ENSURE WITH FIBER consists of oat fiber and soy polysaccharide. This blend results in approximately 4 grams of total dietary fiber per 8-fl. oz can. The ratio of insoluble to soluble fiber is 95:5.

The various nutritional supplements described above and known to others of skill in the art can be substituted and/or supplemented with the PUFAs produced in accordance with the present invention.

J. Oxepa™ Nutritional Product

Oxepa is a low-carbohydrate, calorically dense, enteral nutritional product designed for the dietary management of patients with or at risk for ARDS. It has a unique combination of ingredients, including a patented oil blend containing eicosapentaenoic acid (EPA from fish oil), γ-linolenic acid (GLA from borage oil), and elevated antioxidant levels.

Caloric Distribution:

Caloric density is high at 1.5 Cal/mL (355 Cal/8 fl oz), to minimize the volume required to meet energy needs.

The distribution of Calories in Oxepa is shown in Table A.

TABLE A
Caloric Distribution of Oxepa

| | per 8 fl oz. | per liter | % of Cal |
|---|---|---|---|
| Calories | 355 | 1,500 | — |
| Fat (g) | 22.2 | 93.7 | 55.2 |
| Carbohydrate (g) | 25 | 105.5 | 28.1 |
| Protein (g) | 14.8 | 62.5 | 16.7 |
| Water (g) | 186 | 785 | — |

Fat:

Oxepa contains 22.2 g of fat per 8-fl oz serving (93.7 g/L).

The fat source is an oil blend of 31.8% canola oil, 25% medium-chain triglycerides (MCTs), 20% borage oil, 20% fish oil, and 3.2% soy lecithin. The typical fatty acid profile of Oxepa is shown in Table B.

Oxepa provides a balanced amount of polyunsaturated, monounsaturated, and saturated fatty acids, as shown in Table VI.

Medium-chain trigylcerides (MCTs)—25% of the fat blend—aid gastric emptying because they are absorbed by the intestinal tract without emulsification by bile acids.

The various fatty acid components of Oxepa™ nutritional product can be substituted and/or supplemented with the PUFAs produced in accordance with this invention.

TABLE B
Typical Fatty Acid Profile

| Fatty | | | |
|---|---|---|---|
| Acids | % Total | g/8 fl oz* | 9/L* |
| Caproic (6:0) | 0.2 | 0.04 | 0.18 |
| Caprylic (8:0) | 14.69 | 3.1 | 13.07 |
| Capric (10:0) | 11.06 | 2.33 | 9.87 |
| Palmitic (16:0) | 5.59 | 1.18 | 4.98 |
| Palmitoleic | 1.82 | 0.38 | 1.62 |
| Stearic | 1.94 | 0.39 | 1.64 |
| Oleic | 24.44 | 5.16 | 21.75 |
| Linoleic | 16.28 | 3.44 | 14.49 |
| α-Linolenic | 3.47 | 0.73 | 3.09 |
| γ-Linolenic | 4.82 | 1.02 | 4.29 |
| Eicosapentaenoic | 5.11 | 1.08 | 4.55 |
| n-3-Docosapent- | 0.55 | 0.12 | 0.49 |
| aenoic | | | |
| Docosahexaenoic | 2.27 | 0.48 | 2.02 |
| Others | 7.55 | 1.52 | 6.72 |

Fatty acids equal approximately 95% of total fat.

TABLE C
Fat Profile of Oxepa.

| % of total calories from fat | 55.2 | |
|---|---|---|
| Polyunsaturated fatty acids | 31.44 | g/L |
| Monounsaturated fatty acids | 25.53 | g/L |
| Saturated fatty acids | 32.38 | g/L |
| n-6 to n-3 ratio | 1.75:1 | |
| Cholesterol | 9.49 | mg/8 fl oz |
| | 40.1 | mg/L |

Carbohydrate:

The carbohydrate content is 25.0 g per 8-fl-oz serving (105.5 g/L).

The carbohydrate sources are 45% maltodextrin (a complex carbohydrate) and 55% sucrose (a simple sugar), both of which are readily digested and absorbed.

The high-fat and low-carbohydrate content of Oxepa is designed to minimize carbon dioxide (CO2) production. High CO2 levels can complicate weaning in ventilator-dependent patients. The low level of carbohydrate also may be useful for those patients who have developed stress-induced hyperglycemia.

Oxepa is lactose-free.

Dietary carbohydrate, the amino acids from protein, and the glycerol moiety of fats can be converted to glucose within the body. Throughout this process, the carbohydrate requirements of glucose-dependent tissues (such as the central nervous system and red blood cells) are met. However, a diet free of carbohydrates can lead to ketosis, excessive catabolism of tissue protein, and loss of fluid and electrolytes. These effects can be prevented by daily ingestion of 50 to 100 g of digestible carbohydrate, if caloric intake is adequate. The carbohydrate level in Oxepa is also sufficient to minimize gluconeogenesis, if energy needs are being met.

Protein:

Oxepa contains 14.8 g of protein per 8-fl-oz serving (62.5 g/L).

The total calorie/nitrogen ratio (150:1) meets the need of stressed patients.

Oxepa provides enough protein to promote anabolism and the maintenance of lean body mass without precipitating respiratory problems. High protein intakes are a concern in patients with respiratory insufficiency. Although protein has little effect on CO2 production, a high protein diet will increase ventilatory drive.

The protein sources of Oxepa are 86.8% sodium caseinate and 13.2% calcium caseinate.

The amino acid profile of the protein system in

Oxepa meets or surpasses the standard for high quality protein set by the National Academy of Sciences.

* Oxepa is gluten-free.

1. An isolated nucleotide sequence corresponding to or complementary to at least about 50% of the nucleotide sequence selected from the group consisting of SEQ ID NO: 14, SEQ ID NO: 15, SEQ ID NO: 16 and SEQ ID NO: 17.

2. The isolated nucleotide sequence of claim 1 wherein said sequence is selected from the group consisting of SEQ ID NO: 14, SEQ ID NO: 15 and SEQ I6 NO: 17.

3. The isolated nucleotide sequence of claims 1 or 2 wherein said sequence encodes a functionally active desaturase which utilizes a monounsaturated or polyunsaturated fatty acid as a substrate.

4. The nucleotide sequence of claim 1 wherein said sequence is derived from a fungus.

5. The nucleotide sequence of claim 4 wherein said sequence selected from the group consisting of SEQ ID NO: 14, SEQ ID NO: 15, SEQ ID NO: 16 and SEQ ID NO: 17 is derived from the fungus Thraustochytrium aureum.

6. A purified protein encoded by said nucleotide sequence of claims 1 or 2.

7. A purified polypeptide which desaturates polyunsaturated fatty acids at carbon 4 and has at least about 50% amino acid similarity to an amino acid sequence selected from the group consisting of SEQ ID NO: 18, SEQ ID NO: 19, SEQ ID NO: 20 and SEQ ID NO: 21.

8. A method of producing a desaturase comprising the steps of:
a) isolating said nucleotide sequence represented selected from the group consisting of SEQ ID NO: 14, SEQ ID NO: 15, SEQ ID NO: 16 and SEQ ID NO: 17;
b) constructing a vector comprising: i) said isolated nucleotide sequence operably linked to ii) a promoter;
c) introducing said vector into a host cell under time and conditions sufficient for expression of said desaturase.

9. The method of claim 8 wherein said host cell is a eukaryotic cell or a prokaryotic cell.

10. The method of claim 9 wherein said prokaryotic cell is selected from the group consisting of Escherichia coli, Cyanobacteria, and Bacillus subtilis.

11. The method of claim 9 wherein said eukaryotic cell is selected from the group consisting of a mammalian cell, an insect cell, a plant cell and a fungal cell.

12. The method of claim 11 wherein said fungal cell is a yeast cell.

13. The method of claim 12 wherein said yeast cell is selected from the group consisting of Saccharomyces cerevisiae, Saccharomyces carlsbergensis, Candida spp., Lipomyces starkey, Yarrowia lipolytica, Kluyveromyces spp., Hansenula spp., Trichoderma spp. and Pichia spp.

14. A vector comprising: a) a nucleotide sequence selected from the group consisting of SEQ ID NO: 14, SEQ ID NO: 15, SEQ ID NO: 16 and SEQ ID NO: 17, operably linked to b) a promoter.

15. A host cell comprising said vector of claim 14.

16. The host cell of claim 15, wherein said host cell is a eukaryotic cell or a prokaryotic cell.

17. The host cell of claim 16 wherein said prokaryotic cell is selected from the group consisting of Escherichia coli, Cyanobacteria, and Bacillus subtilis.

18. The host cell of claim 16 wherein said eukaryotic cell is selected from the group consisting of a mammalian cell, an insect cell, a plant cell and a fungal cell.

19. The host cell of claim 18 wherein said fungal cell is a yeast cell.

20. The host cell of claim 19 wherein said yeast cell is selected from the group consisting of Saccharomyces cerevisiae, Saccharomyces carisbergensis, Candida spp., Lipomyces starkey, Yarrowia lipolytica, Kluyveromyces spp., Hansenula spp., Trichoderma spp. and Pichia spp.

21. A plant cell, plant or plant tissue comprising said vector of claim 14, wherein expression of said nucleotide sequence of said vector results in production of a polyunsaturated fatty acid by said plant cell or tissue.

22. The plant cell, plant or plant tissue of claim 21 wherein said polyunsaturated fatty acid is selected from the group consisting of ω6-docosapentaenoic acid and docosahexaenoic acid.

23. One or more plant oils or acids expressed by said plant cell, plant or plant tissue of claim 22.

24. A transgenic plant comprising said vector of claim 14, wherein expression of said nucleotide sequence of said vector results in production of a polyunsaturated fatty acid in seeds of said transgenic plant.

25. A transgenic, non-human mammal whose genome comprises a nucleotide sequence encoding Δ4-desaturase, operably linked to a promoter.

26. The transgenic, non-human mammal of claim 25, wherein said nucleotide sequence is selected from the group consisting of SEQ ID NO: 14, SEQ ID NO: 15, SEQ ID NO: 16 and SEQ ID NO: 17.

27. A fluid produced by or tissue of said transgenic, non-human mammal of claim 26 wherein said fluid or tissue comprises a detectable level of at least Δ4-desaturase when said nucleotide sequence is selected from the group consisting of SEQ ID NO: 14, SEQ ID NO: 15, SEQ ID NO: 16 and SEQ ID NO: 17.

28. A method for producing a polyunsaturated fatty acid comprising the steps of:
a) isolating a nucleotide sequence selected from the group consisting of SEQ ID NO: 14, SEQ ID NO: 15, SEQ ID NO: 16 and SEQ ID NO: 17;
b) constructing a vector comprising said isolated nucleotide sequence;
c) introducing said vector into a host cell under time and conditions sufficient for expression of Δ4-desaturase; and
d) exposing said expressed Δ4-desaturase to a substrate polyunsaturated fatty acid in order to convert said substrate to a product polyunsaturated fatty acid.

29. The method according to claim 28, wherein said substrate polyunsaturated fatty acid is adrenic acid or ω3-docosapentaenoic acid and said product polyunsaturated fatty acid is ω6-docosapentaenoic acid or docosahexaenoic acid, respectively.

30. The method according to claim 29 further comprising the step of exposing said product polyunsaturated fatty acid to a desaturase in order to convert said product polyunsaturated fatty acid to another polyunsaturated fatty acid.

31. The method according to claim 30 wherein said product polyunsaturated fatty acid is ω6-docosapentaenoic acid and said another polyunsaturated fatty acid is docosahexaenoic acid.

32. A method of producing a polyunsaturated fatty acid comprising the steps of:
a) exposing a substrate polyunsaturated fatty acid to one or more enzymes selected from the group consisting of a desaturase and an elongase in order to convert said substrate to a product polyunsaturated fatty acid; and
b) exposing said product polyunsaturated fatty acid of step (a) to a Δ4-desaturase comprising an amino acid sequence selected from the group consisting of SEQ ID NO: 18, SEQ ID NO: 19, SEQ ID NO: 20 and SEQ ID NO: 21, in order to convert said product polyunsaturated fatty acid to a final product polyunsaturated fatty acid.

33. The method of claim 32 wherein said substrate polyunsaturated fatty acid is selected from the group consisting of linoleic acid, α-linolenic acid, stearidonic acid, arachidonic acid, dihomo-γ-linolenic acid, ω6-docosapentaenoic acid and eicosapentaenoic acid.

34. The method of claim 32 wherein said final product polyunsaturated fatty acid is selected from the group consisting of eicosapentaenoic acid, ω6-docosapentaenoic acid and docosahexaenoic acid.

35. A nutritional composition comprising at least one polyunsaturated fatty acid selected from the group consisting of said product polyunsaturated fatty acid produced according to the method of 28 and said another polyunsaturated fatty acid produced according to the method of claim 30.

36. The nutritional composition of claim 35 wherein said product polyunsaturated fatty acid is at least one polyunsaturated fatty acid selected from the group consisting of ω6-docosapentaenoic acid and docosahexaenoic acid.

37. The nutritional composition of claim 35 wherein said another polyunsaturated fatty acid is docosahexaenoic acid.

38. The nutritional composition of claim 35 wherein said nutritional composition is selected from the group consisting of an infant formula, a dietary supplement and a dietary substitute.

39. The nutritional composition of claim 35 wherein said nutritional composition is administered to a human or an animal.

40. The nutritional composition of claim 36 wherein said nutritional composition is administered enterally or parenterally.

41. The nutritional composition of claim 35 wherein said nutritional composition further comprises at least one macronutrient selected from the group consisting of coconut oil, soy oil, canola oil, monoglycerides, diglycerides, glucose, edible lactose, electrodialysed whey, electrodialysed skim milk, milk whey, soy protein, and protein hydrolysates.

42. The nutritional composition of claim 41 wherein said nutritional composition further comprises at least one vitamin selected from the group consisting of Vitamins A, C, D, E, and B complex and at least one mineral selected from the group consisting of calcium magnesium, zinc, manganese, sodium, potassium, phosphorus, copper, chloride, iodine, selenium and iron.

43. A pharmaceutical composition comprising 1) at least one polyunsaturated fatty acid selected from the group consisting of said product polyunsaturated fatty acid produced according to the method of claim 28 and said another polyunsaturated fatty acid produced according to the method of claim 30 and 2) a pharmaceutically acceptable carrier.

44. The pharmaceutical composition of claim 43 wherein said pharmaceutical composition is administered to a human or an animal.

45. The pharmaceutical composition of claim 44 wherein said pharmaceutical composition further comprises an element selected from the group consisting of a vitamin, a mineral, a carbohydrate, an amino acid, a free fatty acid, a phospholipid, an antioxidant, and a phenolic compound.

46. An animal feed comprising at least one polyunsaturated fatty acid selected from the group consisting of said product polyunsaturated fatty acid produced according to the method of claim 28 and said another polyunsaturated fatty acid produced according to the method of claim 30.

47. The animal feed of claim 46 wherein said product polyunsaturated fatty acid is at least one polyunsaturated fatty acid selected from the group consisting of ω6-docosapentaenoic acid and docosahexaenoic acid.

48. The animal feed of claim 46 wherein said another polyunsaturated fatty acid is docosahexaenoic acid.

49. A method of preventing or treating a condition caused by insufficient intake of polyunsaturated fatty acids comprising administering to said patient said nutritional composition of claim 32 in an amount sufficient to effect said treatment.
